(19) United States
(12) Patent Application Publication
(10) Pub. No.: US 2010/0272745 A1
(43) Pub. Date: Oct. 28, 2010

(54) VACCINES FOR MALARIA

(76) Inventors: Dominique Ingrid Lemoine, Rixensart (BE); Florence Emilie Jeanne Francoise Wauters, Rixensart (BE)

Correspondence Address: GlaxoSmithKline;GLOBAL PATENTS -US, UW2220, P. O. BOX 1539, KING OF PRUSSIA, PA, 19406-0939, US

(21) Appl. No.: 12/809,727

(22) PCT Filed: Dec. 18, 2008
(86) PCT No.: PCT/EP08/67925

§ 371 Date: Jun. 21, 2010

Related U.S. Application Data
(60) Provisional application No. 61/015,762, filed on Dec. 21, 2007.

(51) Int. Cl. A61K 39/29; A61P 33/06

(52) U.S. Cl. 424/192.1

(57) ABSTRACT

The present invention relates to a component for a malaria vaccine comprising: a) an immunogenic particle RTS, S and/or b) an immunogenic particle derived from the CS protein of one or more P. vivax strains and S antigen from Hepatitis B and optionally unfused S antigen, or c) an immunogenic particle comprising RTS, CSV-S and optionally unfused S antigen, and d) a stabilizing agent comprising a stabilizing agent with at least one thiol functional group, or mixtures thereof. Methods for preparing the component, its use in medicine, particularly in the prevention of malarial infections, compositions/vaccines containing the component and use of the latter, particularly in therapy are also disclosed.

The present invention relates to a stabilized lipoprotein particle for the treatment of malaria, methods for preparing the same, its use in medicine, particularly in the prevention of malarial infections, compositions/vaccines containing the particle and use of the latter, particularly in therapy.

Malaria, is one of the world's major health problems with more than 2 to 4 million people dying from the disease each year. One of the most prevalent forms of the disease is caused by the protozoan parasite P. vivax, which is found in tropical and sub-tropical regions. Interestingly the parasite can complete its mosquito cycle at temperatures as low as 15 degrees Celsius, which has allowed the disease to spread in temperate climates.

One of the most acute forms of the disease is caused by the protozoan parasite, Plasmodium falciparum (P. falciparum) which is responsible for most of the mortality attributable to malaria.

The life cycle of Plasmodium is complex, requiring two hosts, man and mosquito for completion. The infection of man is initiated by the innoculation of sporozoites into the blood stream through the bite of an infected mosquito. The sporozoites migrate to the liver and there infect hepatocytes where they differentiate, via the exoerythrocytic intracellular stage, into the merozoite stage which infects red blood cells (RBC) to initiate cyclical replication in the asexual blood stage. The cycle is completed by the differentiation of a number of merozoites in the RBC into sexual stage gametocytes, which are ingested by the mosquito, where they develop through a series of stages in the midgut to produce sporozoites which migrate to the salivary gland.

Due to the fact that the disease caused by P. vivax is rarely lethal, efforts to prevent and treat malaria have been focused on the more deadly form of the disease caused by Plasmodium falciparum (P. falciparum).

Although the disease caused by P. vivax does not usually result in death of the patient, due to the volume of cases, which seems to be increasing, the significant impact on the quality of life of the patient, the increasing reports of the severe incidences of the disease resulting in anemia and death, and the economic impact, an effective vaccination for the disease is still required. Furthermore, a single vaccine able to provide protection against both causes of the disease would be advantageous.

A feature of the P. vivax is that some strains are capable of causing delayed infection by remaining latent in the liver before emerging into the peripheral circulation to manifest clinical symptoms. Thus individuals, for example when traveling through an infected area, may be infected and yet may not exhibit symptoms for several months. This has the potential to cause the spread of the disease and for this reason persons traveling to infected areas are not allowed to donate blood for transfusion for a defined period of time after traveling to the infected region.

P. vivax malaria infection remains latent within the liver while the parasite is undergoing pre-erthrocytic shizogony. If the parasite is controlled at this stage, before it escapes the liver, no clinical symptoms of the disease, are observed in the patient.

The sporozoite stage of Plasmodium has been identified as a potential target of a malaria vaccine. Vaccination with deactivated (irradiated) sporozoite has been shown to induce protection against experimental human malaria (Am. J, Trop. Med. Hyg 24: 297-402, 1975). However, it is has not been possible practically and logistically to manufacture a vaccine for malaria for the general population based on this methodology, employing irradiated sporozoites.

The major surface protein of the sporozoite is known as circumsporozoite protein (CS protein). It is thought to be involved in the motility and invasion of the sporozoite during its passage from the initial site of inoculation by the mosquito into the circulation, where it migrates to the liver.

The CS protein of Plasmodia species is characterized by a central repetitive domain (repeat region) flanked by non-repetitive amino (N-terminus) and carboxy (C-terminus) fragments. The central domain of P. vivax is composed of several blocks of a repeat unit, generally of nine tandem amino acids.

In certain Asian strains, after the central repeat region, an additional sequence of approximately 12 amino acids is present (see SEQ ID No 11). The function of the latter is not known. However, it is hypothesized, by some, that said amino acids may be linked to the delayed onset of clinical symptoms of the disease, although this has not been investigated. It is thought that the N-terminus is characterised by a sequence of 5 amino acids known as region I (see SEQ ID No 1). It is also thought that the C-terminus is characterised by comprising a sequence of 12 amino acids known as region II. The latter contains a cell-adhesive motif, which is highly conserved among all malaria CS protein (see SEQ ID No. 2).

Several groups have proposed subunit vaccines based on the circumsporozoite protein. Two of these vaccines based exclusively on the central repeat region underwent clinical testing in the early 1980's; one was a synthetic peptide, the other was a recombinant protein (Ballou et at Lancet: June 6 (1987) page 1277 onwards and Herrington et at Nature 328:257 (1987)). These vaccines were successful in stimulating an anti-sporozoite response. Nonetheless, the magnitude of the response was disappointing, with some vaccinees not making a response at all. Furthermore, the absence of “boosting” of antibody levels after subsequent injections and results of in vitro lymphocyte proliferation assays suggested that T-cells of most of these volunteers did not recognise the immuno-dominant repeat. Furthermore, the efficiency of these two vaccines was marginal with only one vaccinated volunteer failing to develop parasitemia. These vaccines were not pursued any further.

WO 93/10152 and WO 98/05355 describe a vaccine derived from the CS protein of P. falciparum and it seems that there has been some progress made towards the vaccination against P. falciparum using the approach described therein, see also Heppner et al. 2005, Vaccine 23, 2243-50.

To date the most advanced malaria vaccine in the clinic is based on a lipoprotein particle (also known as a virus like particle) referred to as RTS,S. This particle contains a portion of the CS protein of P. falciparum substantially as corresponding to amino acids 207-395 of the CS protein of P. falciparum (strain NF54/3D7) fused in frame via a linear linker to the N-terminal of the S antigen from Hepatitis B. The linker may comprise a portion of preS2 from the S-antigen. See discussion below for further detail.

The CS protein in P. falciparum has a central repeat region that is conserved. In contrast at least two forms (designated VK210 or type I and VK247 or type II) of the CS protein for P. vivax are known. This renders it more difficult to identify a construct of the CS protein with all the desired properties such as immogenicity, which provides general protection against P. vivax regardless of the specific type of CS protein because antibodies directed the central repeating region of type I do not necessarily recognize epitopes on the corresponding region of type II and vice versa.

As far as the inventors are aware a particle corresponding to RTS,S has not been proposed based on a single strain of P. vivax.

A hybrid P. vivax CS protein is described in WO 2006/088597.

A fusion protein (referred to herein as CSV-S) comprising the hybrid protein of WO 2006/088597 and S antigen from Hepatitis B and lipoprotein particles comprising same are described in PCT/EP2007/057301.

A lipoprotein particle comprising CSV-S, RTS and optionally S units is described in PCT/EP2007/057296.

At the present time RTS,S malaria vaccines are provided as lyophilized antigen, which are reconstituted with adjuvant shortly before delivery. This is because the antigen is unstable when stored for substantial periods of time, particularly in the presence of the adjuvant. The instability manifests itself as agglomeration and/or degradation.

There are estimates by the year 2018/2019 that 83 million doses of malaria vaccines will be required. The current freeze-drying (lyophilization) process takes around 40 hours. Therefore it is unlikely that the present process will be able to meet future needs. It may be possible to reduce the cycle down to about 28 hours but this is still unlikely to meet the need. Furthermore, reducing the cycle time further seems to lead to an unsatisfactory product.

Malaria vaccines are predominantly for delivery in countries with poor infra-structure and facilities, therefore it is vitally important that the form the vaccine is provided in, is stable until administration, especially if a liquid formulation is provided.

Preliminary data generated by the inventors showed that RTS,S purified bulk prepared in phosphate buffered saline and containing a residual amount of polysorbate 80 (0.0062% w/w) with no additional excipients at pH 6.1 showed significant degradation and oxidative aggregation after accelerated stability testing, namely storage for 7 days at 37° C. Slight aggregation and degradation was observed after 2 months storage at 4° C.

The Following Options were Investigated:
pH increased from 6.1 to 7.4 seemed to reduce S antigen degradation but increase CS protein degradation;
an increase in polysorbate 80 (also referred to as Tween 80) concentration to 0.05, 0.5 and 1.0% seemed to increase both aggregation and degradation, which is surprising because normally Tween 80 decreases the aggregation of the antigen (It is hypothesized by the inventors that this was due to the presence of residual peroxide in the Tween which may catalyse oxidation of thiol groups in the protein/antigen-using a reducing agent according to the invention seems to prevent this effect); and
the addition of sucrose (6.2% w/w) had no impact on aggregation or degradation.

It is hypothesized that the aggregation process occurs in a number of stages and that if one of these stages can be successfully prevented then the aggregation and/or degradation can be prevented (reference: “Minimizing protein inactivation” by D. B. Volkin & A. M. Klibanov in “Protein function: a practical approach”, edited by T. E. Creighton—IRL Press at Oxford University Press).

The first stage is unfolding of the native protein, thereby exposing more hydrophobic regions thereof. This exposure of hydrophobic regions results in grouping of several proteins together. The final stage is irreversible denaturing of the protein by the formation of disulphide bonds.

It may also be that polysorbate 80, which is added to solubilise the antigen contains residual peroxide that catalyses aggregation and/or degradation.

The inventors tried a number of stabilizing agents/methods, for example sugars, polyalcohols, co-solvents, polymers, ions, pH, buffers, antioxidants, chelating agents and surfactants, which did not provide the desired effect. For example the addition of ascorbic acid produced significant aggregation. The use of EDTA alone or in the presence of an antioxidant did not prevent aggregation. Furthermore, the addition of sulphite did not provide the required stabilization. Some common stabilizing agents were not compatible with the adjuvant formulation employed in the final malaria formulation. The inventors now believe that the lipoprotein particles of Plasmodium CS protein (be that falciparum and/or vivax) may be stabilized for storage employing specific stabilizing agents, for example reducing agents which contain at least one thiol (—SH) group, such as, thiosulfate, N-acetyl cysteine, monothioglycerol, cysteine, reduced glutathione and sodium thioglycolate or mixtures thereof, particularly N-acetyl cysteine, monothioglycerol, cysteine, sodium thioglycolate and mixtures thereof, especially monothioglycerol, cysteine, and mixtures thereof.

Alternatively or in combination with these reducing agents, which contain at least one thiol (—SH) group, the lipoproteins particles employed in the invention may be stablised or further stabilized by removing oxygen from the container they are stored in and/or protecting the formulation from light (for example by using amber glass containers) may protect/further protect the antigen.

Thus the invention provides a component for a malaria vaccine comprising:
a) an immunogenic particle RTS,S and/or
b) an immunogenic particle derived from the CS protein of one or more P. vivax strains and S antigen from Hepatitis B and optionally unfused S antigen, and/or
c) an immunogenic particle comprising RTS, CSV-S and optionally unfused S antigen, and
d) a stabilizing agent comprising (or selected from the group consisting of) a reducing agent with at least one thiol functional group, for example as listed above such as monothioglycerol, cysteine, N-acetyl cysteine or mixtures thereof.

In one aspect the invention provides a component for a malaria vaccine comprising a), b), c) and optionally d) above and wherein protective measures are employed in the preparation of same such as removing oxygen from the container and/or protecting the formulation from light by, for example using amber glass containers.

Advantageously lipoprotein particle antigens comprising CS protein from Plasmodium and S antigen from Hepatitis may be adequately stabilised employing monothioglycerol, cysteine or mixtures thereof and/or protective measures such as removing oxygen from the vials and/or protecting the formulation against light by using, for example amber glass containers.

### Sequence Listing

SEQ. ID. No. 1 Region I in the N-terminus of P. Vivax
SEQ. ID. No. 2 Is a highly conserved portion of Region II in the C-terminus of P. Vivax
SEQ. ID. No. 3-9 Various repeat units of type I CS protein of P. Vivax
SEQ. ID. No. 10 Major repeat unit from type II CS protein of P. Vivax
SEQ. ID. No. 11 Additional amino acids found in Asian strains of P. Vivax
SEQ. ID. No. 12 Nucleotide sequence of the hybrid protein CSV of P. Vivax (optimized for expression in E. Coli)
SEQ. ID. No. 13 Amino acid sequence of the hybrid protein CSV of P. Vivax
SEQ. ID. No. 14 Minor repeat unit from type II CS protein of P. Vivax
SEQ. ID. No. 15 Nucleotide sequence for the hybrid protein CSV of P. Vivax (optimized for expression in yeast)
SEQ. ID. No. 16 Nucleotide sequence for the hybrid fusion protein CSV-S
SEQ. ID. No. 17 Amino acid sequence for the hybrid fusion protein CSV-S
SEQ. ID No. 18 Nucleotide Sequence for an RTS expression cassette.
SEQ. ID No. 19 Predicted RTS fusion protein from SEQ ID No. 18.
SEQ ID Nos. 20 to 25 Examples of CpG containing oligonucleotides.

### FIGURES

FIG. 1 Plasmid map for pRIT15546a yeast episomal vector.

FIG. 2 Plasmid map of pGF1-S2a plasmid prepared by GSK employed in “fusing” the desired antigen with the S antigen from Hepatitis B. Cloning heterologous DNA sequences between SmaI sites (after excision of the 12 bp SmaI DNA fragment) creates in-frame fusion with the S gene.

FIG. 3 Plasmid map of pRIT15582
Digestion with XhoI liberates a 8.5 kb linear DNA fragment carrying the CSV-S expression cassette plus the LEU2 selective marker, being used for insertion into the yeast chromosome.

FIG. 4 Restriction map of the linear XhoI fragment used to integrate CSV-S cassette

FIG. 5 Electron micrograph of CSV-S,S mixed particles produced in strain Y1835
CSV-S,S particles were purified from soluble cell extracts (based on RTS,S purification process) and submitted to electron microscopy analysis. Particles were visualized after negative staining with phosphotungstic acid. The scale is equivalent to 100 nm.

FIG. 6 Shows SDS page analysis after storage for 7 days at 37° C.+/−AOT—Novex gels in non-reducing (left) and reducing (right) conditions, before (above) or 24 h 25° C. after (below) mixing with AS01, where:
1. Mw
2. PB T0
3. PB 7d 37° C.
4. NaCl PO₄ 7d 37° C.
5. NaCl PO₄ 7d 37° C.+AOT amber glass
6. NaCl PO₄ 7d 37° C.+AOT white glass
7. MTG 0.01% 7d 37° C.
8. MTG 0.01% 7d 37° C.+AOT amber glass
9. MTG 0.01% 7d 37° C.+AOT white glass
10. MTG 0.04% 7d 37° C.
11. MTG 0.04% 7d 37° C.+AOT amber glass
12. MTG 0.04% 7d 37° C.+AOT white glass

FIG. 7 Shows SDS page analysis after storage for 14 days at 37° C.—Novex gel in reducing (left) and non-reducing (right) conditions, before or 24 h 25° C. after mixing with AS01.

FIG. 8 Shows SDS page analysis after storage for 5 weeks at 37° C.—Novex gel in reducing (left) and non-reducing (right) conditions, before or 24 h 25° C. after mixing with AS01

For FIGS. 7 and 8:
1. Mw
2. PB T0 red.
3. RTS,S NaCl PO₄ 5 weeks 37° C. red.
4. RTS,S MTG0.01% 5 weeks 37° C. red.
5. RTS,S MTG0.04% 5 weeks 37° C. red.
6. (RTS,S NaCl PO4 5 weeks 37° C.)/AS01E 24 h 25° C. red.
7. (RTS,S MTG0.01% 5 weeks 37° C.)/AS01E 24 h 25° C. red.
8. (RTS,S MTG0.04% 5 weeks 37° C.)/AS01E 24 h 25° C. red.
9. PB T0 non-red
10. RTS,S NaCl PO4 5 weeks 37° C. non-red.
11. (RTS,S MTG0.01% 5 weeks 37° C. non-red.
12. (RTS,S MTG0.04% 5 weeks 37° C. non-red.
13. (RTS,S NaCl PO4 5 weeks 37° C.)/AS01E 24 h 25° C. red.
14. (RTS,S MTG0.01% 5 weeks 37° C.)/AS01E 24 h 25° C. non-red.
15. (RTS,S MTG0.04% 5 weeks 37° C.)/AS01E 24 h 25° C. non-red.

FIG. 9 Shows RTS,S antigenicity in liquid formulations with or without monothioglycerol by mixed ELISA αCSP-α-S

FIG. 10 Shows anti-CS serology results

FIG. 11 Shows anti-HBS serology results

FIG. 12 Shows CS specific CD4 T cell responses

FIG. 13 Shows HBs specific CD4 T cell responses

FIG. 14 Shows CS specific CD8 T cell responses

FIG. 15 Shows HBs specific CD8 T cell responses

### DETAILED DESCRIPTION OF THE INVENTION

The aspects of the invention that employ N-acetyl cysteine, monothioglycerol, cysteine, reduced glutathione and sodium thioglycolate or mixtures thereof have a further advantage in that this embodiment provides a viable manufacturing alternative to sodium sulfate, (use of which it may be desirable to avoid).

Whilst not wishing to be bound by theory it is hypothesised by the inventors that a thiol function in the stabilizing agent/reducing agent binds to a thiol function in the antigen thereby blocking the site and preventing bonding/interaction of same with a thiol function on different antigen molecule. Furthermore as the stabilizing agent/reducing agent is relatively small it also thought that the epitopes and particularly conformation epitopes in the antigen are not disrupted and thus the immunogenicity of the antigen is retained and aggregation is prevented.

Alternatively or in addition peroxide in the tween is quenched.

In one aspect of the invention the stabilizing agent is monothioglycerol.

In one aspect of the invention the stabilizing agent is cysteine.

In one aspect of the invention the stabilizing agent is N-acetyl cysteine.

The stabilizing agent may for example be employed in amounts in the range 0.01 to 10% w/v, such as 1 to 5%, 2 to 6%, 4 to 7%, 3 to 8%, such as 0.01 to 1%, 0.2 to 0.4%, 0.1% to 0.5%, 0.3 to 0.8%, 0.6 to 0.9%, for example substantially 0.2, 0.4, 0.5 and 0.8%, or such as 0.01 to 0.1%, 0.01 to 0.02%, 0.01 to 0.05%, 0.01 to 0.08%, 0.02 to 0.05%, 0.02 to 0.08% or 0.05 to 0.08% w/v.

Alternatively, the stabilizing agent may be employed in amounts in the range 0.01 to 10% w/w, such as 1 to 5%, 2 to 6%, 4 to 7%, 3 to 8%, such as 0.01 to 1%, 0.2 to 0.4%, 0.1% to 0.5%, 0.3 to 0.8%, 0.6 to 0.9%, for example substantially 0.2, 0.4, 0.5 and 0.8%, or such as 0.01 to 0.1%, 0.01 to 0.02%, 0.01 to 0.05%, 0.01 to 0.08%, 0.02 to 0.05%, 0.02 to 0.08% or 0.05 to 0.08% w/w.

Suitable amounts of cysteine are in the range 0.1 to 1.0% by weight of the overall formulation. So for example in one human dose of 500 μl the amount of cysteine is in the range 100 μg to 5000 μg such as 500 μg.

In one aspect the invention provides a component for a malaria vaccine comprising:
a) an immunogenic particle RTS,S and/or
b) an immunogenic particle derived from the CS protein of one or more P. vivax strains and S antigen from Hepatitis B and optionally unfused S antigen, and
c) a stabilising agent comprising monothioglycerol.

This aspect of the invention may further employ further protective measures such as removing oxygen from the container/vials and/or protecting the formulation against light by for example using amber glass containers.

Monothioglycerol has the formula HSCH₂CH(OH)CH₂OH and is also known as 3-mercapto-1,2-propanediol or 1-thioglycerol. Suitable amounts for use in the present invention include, but are not limited to, the range 0.01 to 10% such as 0.01 to 1% or 0.01 to 0.1%, 0.01 to 0.02%, 0.01 to 0.05%, 0.01 to 0.08%, 0.02 to 0.05%, 0.02 to 0.08% or 0.05 to 0.08% w/v, for example 0.011, 0.012, 0.013, 0.014, 0.015, 0.016, 0.017, 0.018, 0.019, 0.02, 0.025, 0.04, 0.05 or 0.08% w/v. A single human dose of 250 μl may for example contain 10 to 2500 μg such as 25 to 250 μg of monothioglycerol, for example 50, 125 or 200 μg.

Alternatively, suitable amounts for use in the present invention include, but are not limited to, the range 0.01 to 10% such as 0.01 to 1%, 0.01 to 0.1%, 0.01 to 0.02%, 0.01 to 0.05%, 0.01 to 0.08%, 0.02 to 0.05%, 0.02 to 0.08% or 0.05 to 0.08% w/w, for example 0.011, 0.012, 0.013, 0.014, 0.015, 0.016, 0.017, 0.018, 0.019, 0.02, 0.025, 0.04, 0.05 or 0.08% w/w.

Advantageously, monothioglycerol when used according to the invention seems to be compatible with adjuvant formulations, for example oil in water emulsions or liposomal formulations containing MPL and/or QS21.

Furthermore, monothioglycerol reduces lipoprotein particle aggregation induced by liposomal adjuvant formulations of MPL and QS21, thereby providing a liquid formulation similar to that of purified bulk shortly after preparation.

Purified bulk in the context of this specification refers to purified antigen in bulk quantity, which is more than two doses.

Final bulk in the context of this specification refers to more than one or two doses of purified antigen and excipients, such as phosphate buffered saline, excluding adjuvant components.

RTS,S when formulated at 50 μg/ml with 0.01% monothioglycerol in the absence of adjuvant had a profile after storage at 37° C. for 7 days identical to fresh bulk. 0.01% Monothioglycerol was also sufficient to protect RTS,S from aggregation catalyzed by light.

Nevertheless is it expected to obtain a shelf life of about 2 or 3 years for a liquid formulation of a lipoprotein particle of a Plasmodium CS protein, for example at 100 μg/ml of antigen and for example up to 1.0% w/v such as 0.02, 0.05 or 0.08% of monothioglycerol.

In one aspect of the invention the reducing agent is not dithiotreitol.

Liquid components of the vaccine, including adjuvant components thereof, may require storage at about 4° C.

The formulations of the invention have a pH and osmolality suitable for injection. Suitably, the pH of the liquid formulation is about 6.5 to 7.2 such as about 6.6, 6.7, 6.8, 6.9, 7.0 or 7.1.

The formulations of the invention may further comprise a preservative such as thiomersal, for example when more than 10 doses are provided together. However, in at least one embodiment the formulations described herein are thiomersal free.

Studies indicated that RTS,S, for example at 50 μg/ml stored with 0.01 or 0.04% monothioglycerol after 5 weeks at 4° C. or 37° C. had no detectable antigen loss by non-specific adsorption.

Furthermore, no modification of RTS,S particle size distribution was observed after accelerated stability ie storage for 7 days at 37° C., followed by the exposure to intense light for about 15 hours (referred to herein as accelerated oxidation testing AOT).

In one aspect the invention is provided as a component for a malaria vaccine as a separate liquid formulation and an adjuvant suitable for addition to same, optionally as a kit comprising separate vials of the each element. In one aspect of this embodiment each vial is visually distinct, for example the crimped cap on one vial is coloured to distinguish it from the other vial and/or one vial is amber (such as the antigen containing vial) and one vial is clear (such as the adjuvant containing vial).

Suitable vials include for example 3 mL glass vials.

In one aspect the invention provides lyophilized component containing the antigen and the stabilizing agent (or reducing agent as herein described), which may then be reconstituted with liquid adjuvant. The lyophilized component and the liquid adjuvant (such as an oil in water or liposomal formulation of MPL and QS21) may be provided as a kit. This aspect of the invention has the advantage that it does not need to be used immediately after reconstitution but is stable for storage for at least 24 hours, for example antigenicity of the antigen is maintained for at least 24 hours when stored at 25° C. post mixing. Adjuvants are discussed in detail below.

In one aspect of the invention there is provided a final liquid formulation. Final liquid formulation refers a liquid formulation containing up to 10 doses such as 1 or 2 doses and containing all excipients other than adjuvant components.

In one aspect the component or final vaccine is provided as a single dose.

Vaccine in the context of this specification is the immunogenic formulation containing all the components including adjuvant components suitable for injection into a human.

In one aspect the component or final vaccine is provided as a bidose. This can be beneficial (for example when the quantities for one dose are small) because providing two doses can minimize losses of vital components when reconstituting and/or administering the final formulation.

Thus a vaccine is, for example provided as 2-vial formulation in a bidose presentation comprising:
vial 1: 500 μl (2 doses) of RTS,S 2× concentrated (100 μg/ml)+monothioglycerol (0.02, 0.05 or 0.08%)
vial 2: 500 μl (2 doses) of adjuvant 2× concentrated (AS01)

After reconstitution the formulation provides 1 ml (2 doses) of RTS,S in AS01, +monothioglycerol 0.01, 0.025 or 0.04%.

P. Vivax antigens

CSV-S protein employed in the invention may comprise: a portion derived from the CS protein of P. vivax (CSV). This CSV antigen may a native protein such as found in type I CS proteins of P. vivax and/or as found in type II proteins of P. vivax. Alternatively the CSV protein may be a hybrid protein or chimeric protein comprising elements from said type I and II CS proteins. When the latter is fused to the S antigen this will be referred to herein as a hybrid fusion protein.

CSV-S is used herein as a generic term to cover fusion proteins comprising a sequence/fragment from the CS protein of P. vivax and a sequence from the S-antigen of Hepatitis B.

The hybrid/chimeric protein will generally comprise:

at least one repeat unit derived from the central repeat section of a type I circumsporozoite protein of P. vivax, and
at least one repeat unit derived from the central repeating section of a type II circumsporozoite protein of P. vivax.

Generally the hybrid protein will also contain an N-terminus fragment from CS protein of Plasmodium such as P. vivax, for example a fragment comprising region I such as the amino acids shown in SEQ ID No. 1.

Usually the hybrid protein will contain a C-terminus fragment from CS protein of Plasmodium such as P. vivax, for example a fragment comprising region II such as the motif shown in SEQ ID No 2.

Whilst not wishing to be bound by theory it is thought that the N and C terminal fragments include several T and B cell epitopes.

Any suitable strain of P. vivax may be employed in the invention including: Latina, America (ie Sal 1, Belem), Korean, China, Thailand, Indonesia, India, and Vietnam. The construct in SEQ ID No 13 is based on a Korean strain (more specifically a South Korean strain).

P. vivax with type I CS proteins is more prevalent than P. vivax with type II CS proteins. Therefore in one aspect the invention employs a CS protein from type I. In an alternative aspect the invention provides a hybrid protein comprising a repeat unit from type I and a repeat unit from type II, for example wherein more repeat units from type I are included in the hybrid than repeat units of type II.

More specifically the hybrid protein of the invention may include 1 to 15 repeat units such as 9 repeat units from type I.

Examples of suitable repeat units from type I CS proteins are given in SEQ ID Nos. 3 to 9.

In one embodiment the invention provides a hybrid with a mixture of different repeat units of type I, such as one of each of those listed in SEQ ID Nos. 3 to 9.

One or more repeat units may be duplicated in the hybrid, for example two repeat units of SEQ ID No 3 and/or 4 may be incorporated into the construct.

a) In one aspect the CS protein comprises a unit of SEQ ID No 3.
b) In one aspect the CS protein comprises a unit of SEQ ID No 4, optionally in combination with units as described in paragraph a) directly above.
c) In one aspect the CS protein comprises a unit of SEQ ID No 5, optionally in combination with units as described in paragraph a) or b) directly above.
d) In one aspect the CS protein comprises a unit of SEQ ID No 6, optionally in combination with one or more units as described in paragraphs a) to c) directly above.
f) In one aspect the CS protein comprises a unit of SEQ ID No 7, optionally in combination with one or more units as described in paragraph a) to d) directly above.
g) In one aspect the CS protein comprises a unit of SEQ ID No 8, optionally in combination with one or more units as described in paragraph a) to f) directly above.
h) In one aspect the CS protein comprises a unit of SEQ ID No 9, optionally in combination with one or more units as described in paragraph a) to g) directly above.

Examples of suitable component repeat units from type II CS proteins are given in SEQ ID Nos. 10 and 14, such as 10.

In one aspect of the invention there is provided a hybrid protein with 5 or less repeat units derived from type II such as one repeat unit, for example as shown in SEQ ID No. 10.

The hybrid may also include the 12 amino acid insertion found at the end of the repeat region found in certain Asian strains of P. vivax, for example as shown in SEQ ID No. 11.

In one embodiment the hybrid protein comprises about 257 amino-acids derived from P. vivax CS protein.

The CSV derived antigen component of the invention is generally fused to the amino terminal end of the S protein.

It is believed that the presence of the surface antigen from Hepatitis B boosts the immunogenicity of the CS protein portion, aids stability, and/or assists reproducible manufacturing of the protein.

In one embodiment the hybrid fusion protein comprises about 494 amino acids, for example about 257 of which are derived from P. vivax CS protein.

The hybrid fusion protein may also include further antigens derived from P. falciparium and/or P. vivax, for example wherein the antigen is selected from DBP, PvTRAP, PvMSP2, PvMSP4, PvMSP5, PvMSP6, PvMSP7, PvMSP8, PvMSP9, PvAMA1 and RBP or fragment thereof.

Other example, antigens derived from P. falciparum include, PfEMP-1, Pfs 16 antigen, MSP-1, MSP-3, LSA-1, LSA-3, AMA-1 and TRAP. Other Plasmodium antigens include P. falciparum EBA, GLURP, RAPT, RAP2, Sequestrin, Pf332, STARP, SALSA, PfEXP1, Pfs25, Pfs28, PFS27/25, Pfs48/45, Pfs230 and their analogues in other Plasmodium spp.

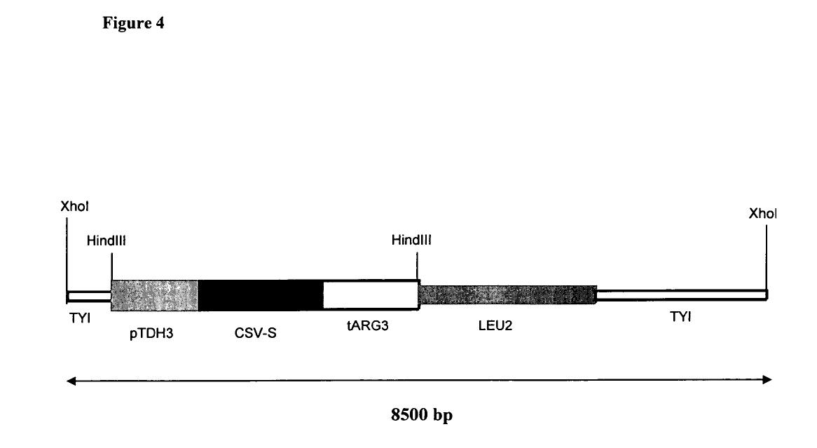

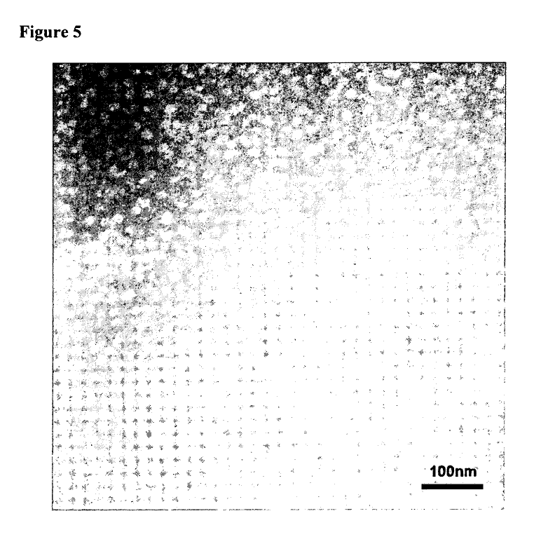

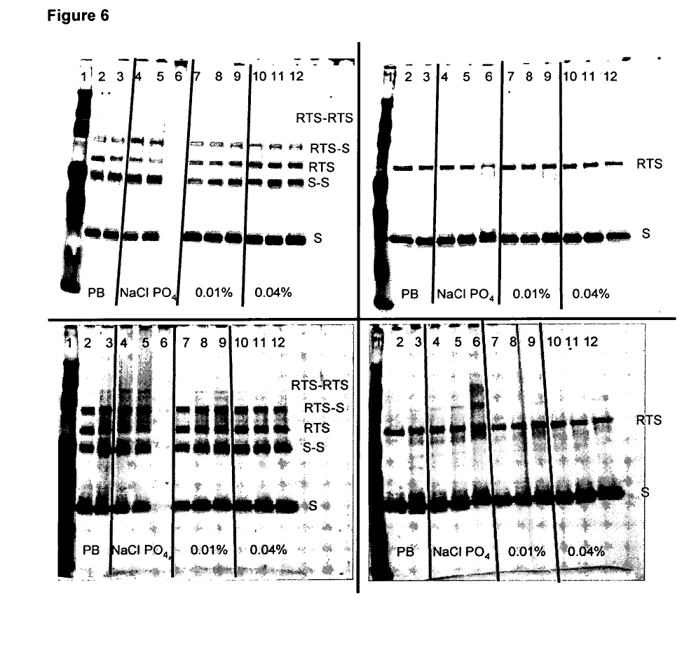

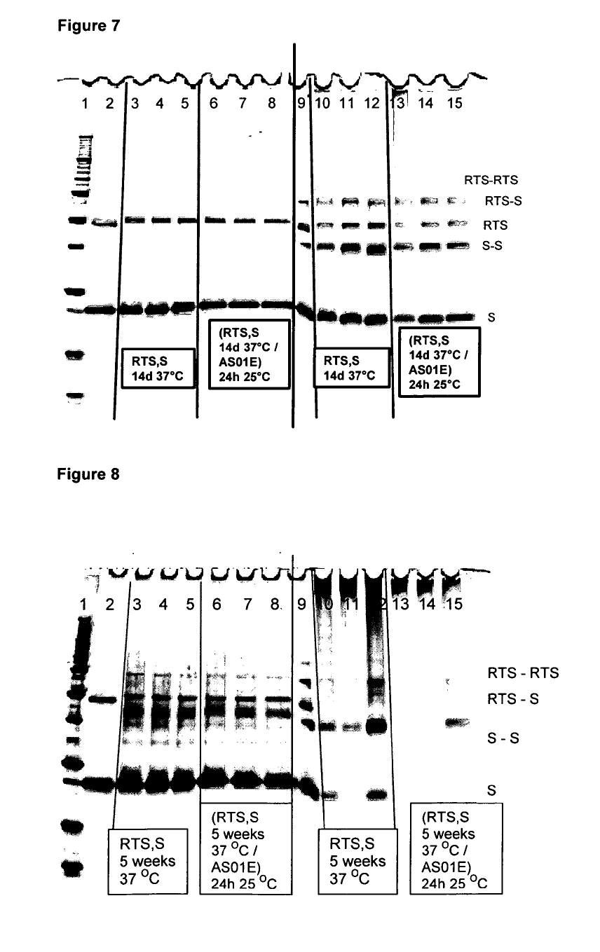

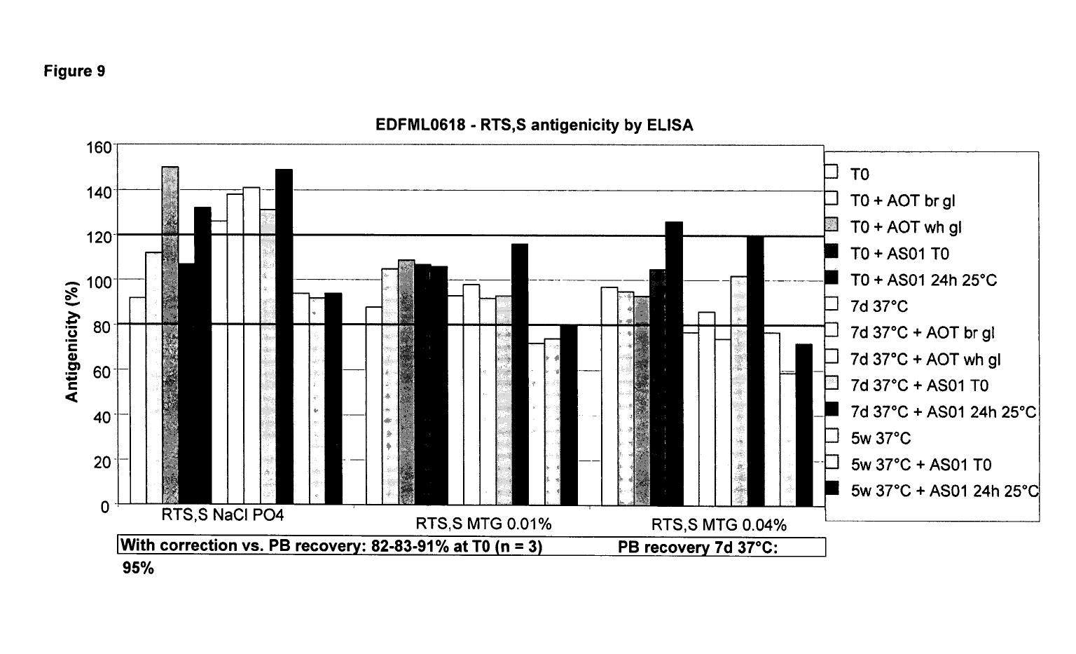

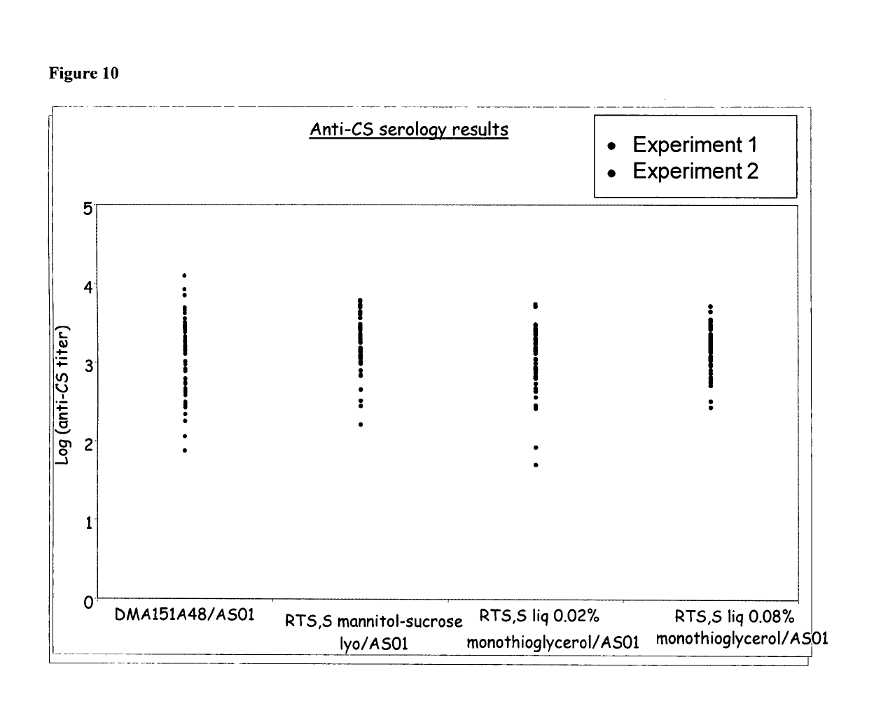

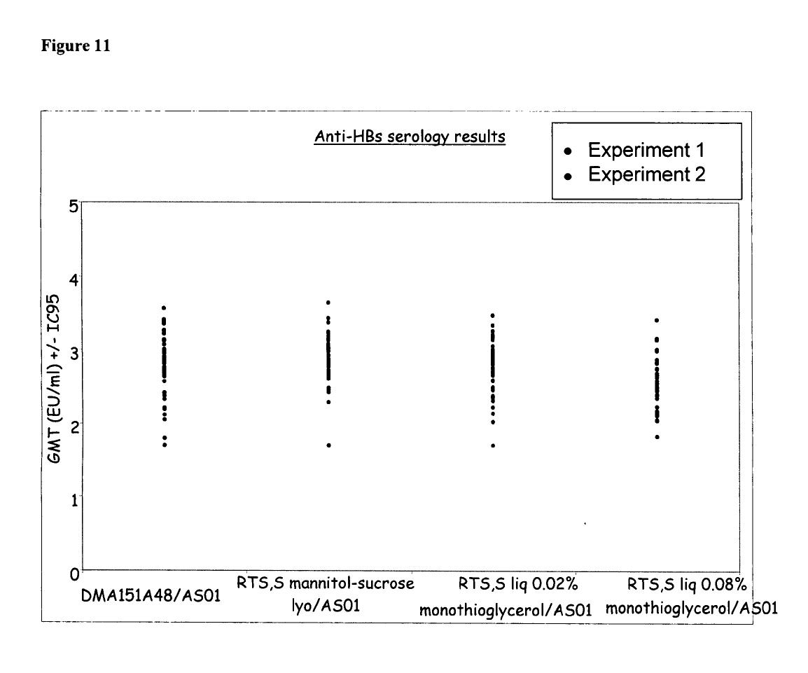

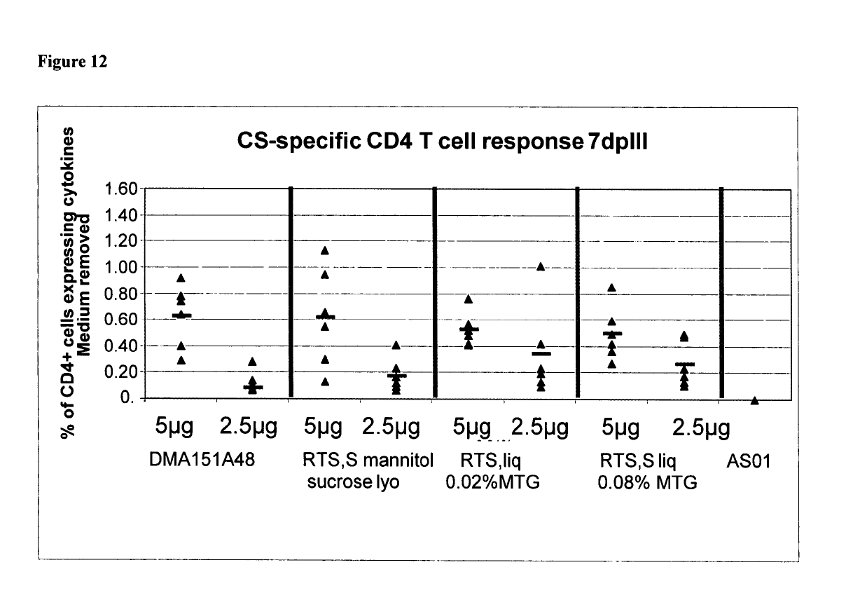

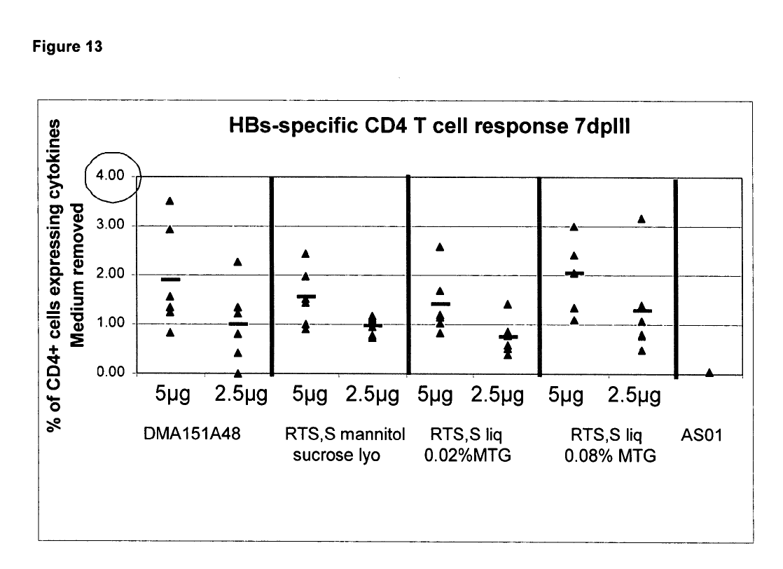

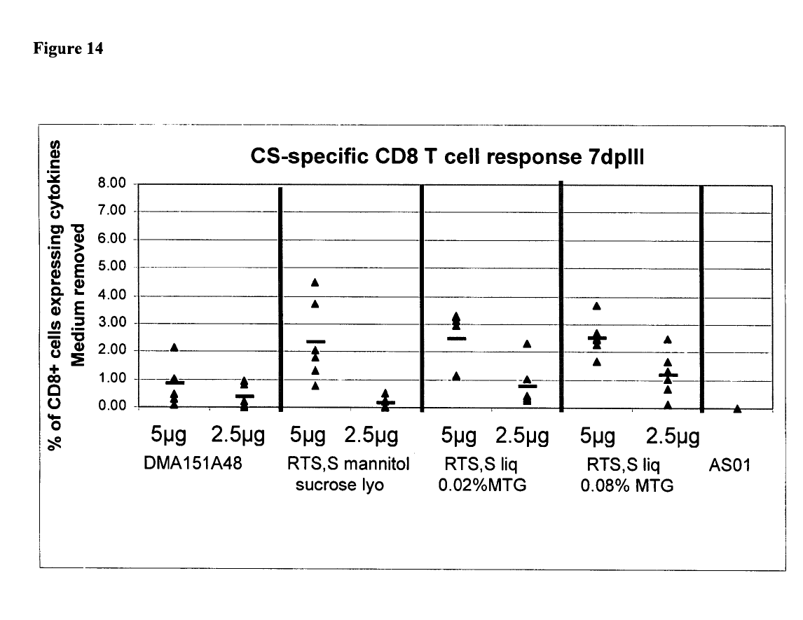

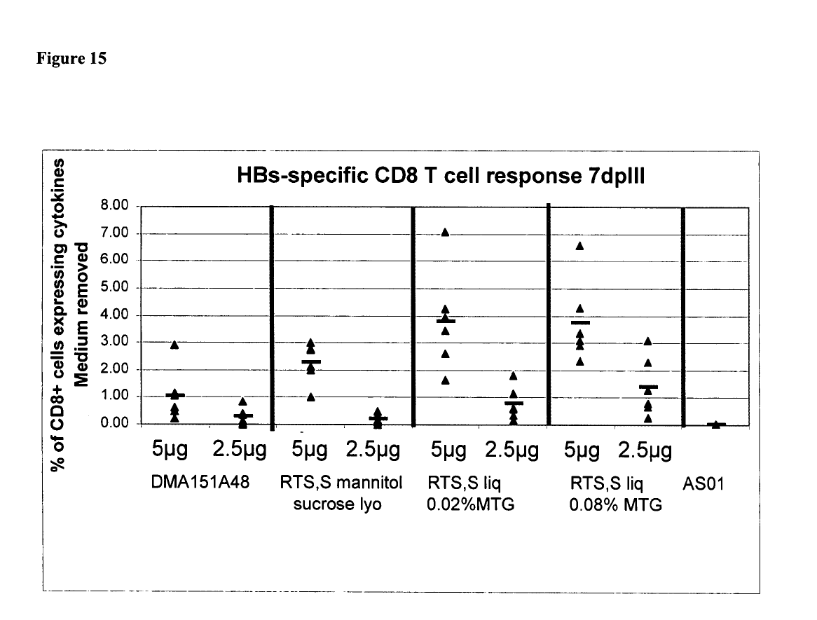

In an embodiment the hybrid fusion protein (CSV-S) has the amino acid sequence shown in SEQ ID No. 17. In the sequence amino acids 6 to 262 are derived from CSV and 269 to 494 are derived from S. The remaining amino acids are introduced by genetic construction (which, in particular may be varied as appropriate). These four amino acids, Met, Met Ala Pro, are derived specifically from plasmid pGF1-S2 (see FIG. 4)

The nucleotide sequence for protein of SEQ ID No 17 is given in SEQ ID No 16.

The polynucleotide sequences which encode immunogenic CS polypeptides may be codon optimised for mammalian cells. Such codon-optimisation is described in detail in WO 05/025614.

### RTS,S

The component of the protein particles of the invention termed RTS (ie derived from P. falciparum) can be prepared as described in WO 93/10152, which includes a description of the RTS* (from P. falciparum NF54/3D7 strain-referred to herein as RTS).

In one or more embodiments of the invention the antigen derived from P. falciparum employed in the fusion protein may be the substantially the whole CS protein thereof.

In one embodiment of the invention full-length S-antigen is employed. In another embodiment a fragment of said S-antigen is employed.

In one embodiment the antigen derived from of P. falciparum comprises at least 4 repeat units the central repeat region. More specifically this antigen comprises a sequence which contains at least 160 amino acids, which is substantially homologous to the C-terminal portion of the CS protein. The CS protein may be devoid of the last 12 to 14 (such as 12) amino-acids from the C terminal.

More specifically the fusion protein derived from P. falciparium employed is that encoded for by the nucleotide sequence for the RTS expression cassette, provide in SEQ ID No 18.

S-Antigen from Hepatitis B

Suitable S antigens may comprise a preS2 region. An example of a suitable serotype is adw (Nature 280:815-819, 1979).

Usually the sequence from Hepatitis B will be full length S-antigen. Generally the preS2 region will not be included.

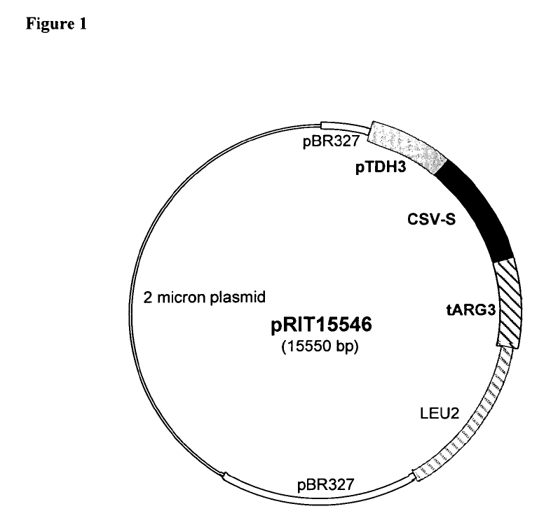

In one aspect the hybrid fusion proteins of the invention comprise a portion derived from a mutant S protein, for example as described in published US application No. 2006/194196 (also published as WO 2004/113369). This document describes a mutant labeled HDB05. In particular it describes comparisons of the mutant and wild type proteins in FIGS. 1 and 6 and genes for the mutant in FIGS. 4 and 5. Sequence 12 to 22 therein describe particular polypeptides of the mutant S protein. Each of the above is incorporated herein by reference.

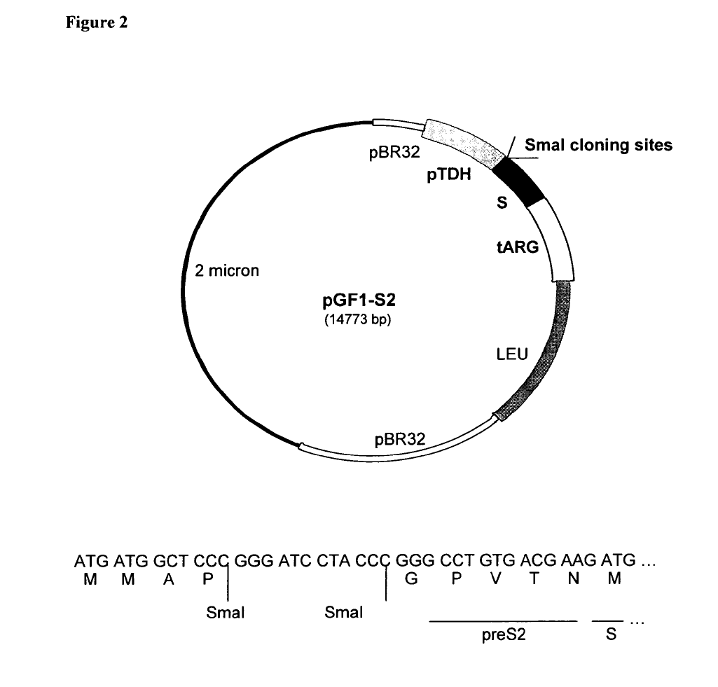

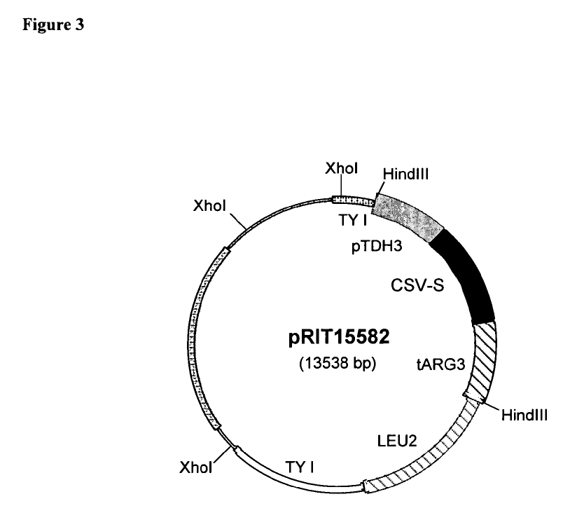

The fusion protein CSV-S may for example be prepared employing the plasmid pGF1-S2 (see FIG. 2 and the examples for further details), which when the appropriate sequence corresponding to CSV is inserted at the SamI cloning site can under suitable conditions produce the fusion protein CSV-S.

The DNA sequences encoding the proteins of the present invention may be flanked by transcriptional control elements, preferably derived from yeast genes and incorporated into an expression vector.

An expression cassette for hybrid proteins employed in the invention may, for example, be constructed comprising the following features:
A promoter sequence, derived, for example, from the S. cerevisiae TDH3 gene.
A sequences encoding for an appropriate fusion protein.
A transcription termination sequence contained within the sequence, derived, for example, from the S. cerevisiae ARG3 gene.

An example of a specific promoter is the promoter from the S. cerevisiae TDH3 gene Musti et al.

A suitable plasmid can then be employed to insert the sequence encoding for the hybrid fusion protein into a suitable host for synthesis. An example of a suitable plasmid is pRIT15546a 2 micron-based vector for carrying a suitable expression cassette, see FIG. 1 and Examples for further details.

The plasmid will generally contain an in-built marker to assist selection, for example a gene encoding for antibiotic resistance or LEU2 or HIS auxotrophy.

Generally the host will have an expression cassette for each fusion protein in the particle and may also have one or more expression cassettes for the S antigen integrated in its genome.

The invention also relates to a host cell transformed with a vector according to the invention. Host cells can be prokaryotic or eukaryotic but preferably, are yeast, for example Saccharomyces (for example Saccharomyces cerevisiae such as DC5 in ATCC data base (accession number 20820), under the name RIT DC5 cir(o). Depositor: Smith Kline-RIT) and non-Saccharomyces yeasts. These include Schizosaccharomyces (eg Schizosaccharomyces pombe) Kluyveromyces (eg Kluyveromyces lactis), Pichia (eg Pichia pastoris), Hansenula (eg Hansenula polymorpha), Yarrowia (eg Yarrowia lipolytica) and Schwanniomyces (eg Schwanniomyces occidentalis).

A suitable recombinant yeast strain is Y1834 (and use thereof forms part of the invention) for expressing the fusion protein, see Examples for preparation of the same.

The nucleotide sequences or part thereof (such as the portion encoding the CS/hybrid protein but optionally not the portion encoding protein S) employed herein may be codon-optimized for expression in a host, such as yeast.

The host cell may comprise an expression cassette for a fusion protein derived from P. vivax and an expression cassette for the fusion protein derived from P. falciparum and optionally S antigen.

In certain hosts, such as yeast cells, once expressed the fusion protein (comprising the S antigen) is spontaneously assembled into a protein structure/particle composed of numerous monomers of said fusion proteins. When the yeast expresses two different fusion proteins (or a fusion(s) protein and S antigen) these are believed to be co-assembled in particles.

When the chosen recipient yeast strain already carries in its genome several integrated copies of Hepatitis B S expression cassettes then the particles assembled may also include monomers of unfused S antigen.

These particles may also be referred to a Virus Like Particles (VLP). The particles may also be described as multimeric lipoprotein particles, or simply as immunogenic particles.

Thus there is provided an immunogenic protein particle comprising the following monomers:
a. a fusion protein comprising sequences derived from a CS protein of P. vivax, (such as CSV-S) and/or
b. a fusion protein comprising sequences derived from CS protein of P. falciparum (such as RTS), and
c. optionally unfused S antigen
   wherein said particle(s) is/are in association with a stabilizing agent for example as defined above such as monothioglycerol, cysteine or mixtures thereof,

In one aspect the invention provides an immunogenic protein particle comprising the monomers a) and/or b) and c) as defined above and protective wherein the oxygen has been removed from a container or vial holding the particles and/or wherein the particle(s) is/are protect from light, for example by amber glass containers.

In a further aspect the invention provides use of a fusion protein comprising:
a) a sequence derived from a CS protein of P. vivax (such as a sequence from the repeat region of type I and/or type II)
b) a sequence derived from the CS protein of P. falciparum (such as a sequence from the repeat region thereof), and
c) a sequence from the S-antigen of Hepatitis B
   which when expressed in a suitable host provides virus like particles comprising the fusion protein and optionally unfused S antigen to produce particle(s) in association with a reducing agent as defined herein, for example selected from monothioglycerol, cysteine or mixtures thereof.

In a further aspect the invention provides use of a fusion protein comprising:
a) a sequence derived from a CS protein of P. vivax (such as a sequence from the repeat region of type I and/or type II)
b) a sequence derived from the CS protein of P. falciparum (such as a sequence from the repeat region thereof), and
c) a sequence from the S-antigen of Hepatitis B
   which when expressed in a suitable host provides virus like particles comprising the fusion protein and optionally unfused S antigen to produce particle(s) in an environment wherein the oxygen has been removed and/or the protein/particle(s) is/are protected from light by, for example using amber glass containers.

Thus the invention extends to use of a reducing agent with at least one thiol functional group, for example as described herein such as monothioglycerol, cysteine or mixtures thereof and particularly monothioglycerol to stabilize a protein particle comprising a fusion protein derived from CS protein of P. vivax and/or a fusion protein derived from CS protein of P. falciparium (such as RTS) in the form of immunogenic lipoprotein particles.

Thus the invention provides use of a reducing agent with at least one thiol functional group, for example as described herein such as monothioglycerol to stabilize a VLP comprising CSV-S and/or RTS units. In one aspect the invention provides a particle consisting essentially of CSV-S and/or RTS units. In an alternative aspect the particles produced comprise or consist of essentially of CSV-S and/or RTS and S units.

It is hypothesized that the lipoprotein particles employed in the invention may contribute to further stimulating in vivo the immune response to the antigenic protein(s).

It is further hypothesized that the addition stabilizing agent with at least one thiol functional group, for example as described herein such as monothioglycerol, cysteine and mixtures provide internal stabilization to each particle and thus the agent may become associated or internalized within a given particle.

The present invention also relates to vaccines comprising an immunoprotective amount of a stabilized protein particle according to the invention in admixture with a suitable excipient for example a diluent.

Vaccine in the context of the present specification refers to a formulation containing all the components including adjuvant components and suitable for injection into a human patient.

Stabilized in the context of the present invention is intended to mean by reference to a corresponding formulation wherein a stabilizing agent (also referred to herein as a reducing agent) with at least one thiol functional group, for example as described herein such as monothioglycerol, cysteine and mixtures thereof, are omitted, for example when stored for 7 or 14 days at 37° C. and/or when stored under accelerated stability conditions such as 7 days a 37° C. followed by treatment for about 15 hours in the presence of intense light.

Stability may be with reference to particle size (as for example measure by light scattering techniques, Size Exclusion Chromatography or Field Flow Fractionation) and/or aggregation/degradation (as for example measure by SDS-page and Western Blot) and/or antigenicity (as for example measured by ELISA) and/or immunogenicity (as for example measured in vivo).

In one aspect stability refers to the absence of aggregation and degradation.

### Compositions

In the context of this specification excipient, refers to a component in a pharmaceutical formulation with no therapeutic effect in its own right. Adjuvant is an excipient because although there may be a physiological effect produced by the adjuvant in the absence of the therapeutic component such as antigen this physiological effect is non-specific and is not therapeutic in its own right. A diluent or liquid carrier falls within the definition of an excipient.

Immunogenic in the context of this specification is intended to refer to the ability to elicit a specific immune response to the CS portion and/or the S antigen portion of the fusion protein employed. This response may, for example be when the lipoprotein particle is administered in an appropriate formulation which may include/require a suitable adjuvant. A booster comprising a dose similar or less than the original dose may be required to obtain the required immunogenic response.

The composition/pharmaceutical formulations according to the invention may also include in admixture one or more further antigens such as those derived from P. falciparium and/or P. vivax, for example wherein the antigen is selected from DBP, PvTRAP, PvMSP2, PvMSP4, PvMSP5, PvMSP6, PvMSP7, PvMSP8, PvMSP9, PvAMA1 and RBP or fragment thereof.

Other example, antigens derived from P. falciparum include, PfEMP-1, Pfs 16 antigen, MSP-1, MSP-3, LSA-1, LSA-3, AMA-1 and TRAP. Other Plasmodium antigens include P. falciparum EBA, GLURP, RAPT, RAP2, Sequestrin, Pf332, STARP, SALSA, PfEXP1, Pfs25, Pfs28, PFS27/25, Pfs48/45, Pfs230 and their analogues in other Plasmodium spp.

The compositions/pharmaceutical formulations according to the invention may also comprise particles of RTS, S (as described in WO 93/10152) in admixture with the particles comprising CSV-S.

In the vaccine of the invention, an aqueous solution of the particle may be used directly. Alternatively, the protein with or without prior lyophilization can be mixed or absorbed with an adjuvant.

### Adjuvants

Suitable adjuvants are those selected from the group of metal salts, oil in water emulsions, Toll like receptors agonist, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof with the proviso that metal salts are only used in combination with another adjuvant and not alone unless they are formulated in such a way that not more than about 60% of the antigen is adsorbed onto the metal salt. In one embodiment the adjuvant does not include a metal salt as sole adjuvant. In one embodiment the adjuvant does not include a metal salt.

In an embodiment the adjuvant is a Toll like receptor (TLR) 4 ligand, for example an agonist such as a lipid A derivative particularly monophosphoryl lipid A or more particularly 3-deacylated monophoshoryl lipid A (3D—MPL).

3-Deacylated monophosphoryl lipid A is known from U.S. Pat. No. 4,912,094 and UK patent application No. 2,220,211 (Ribi) and is available from Ribi Immunochem, Montana, USA.

3D—MPL is sold under the trademark MPL® by Corixa corporation and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3D-MPL is used. Small particle 3D-MPL has a particle size such that it may be sterile-filtered through a 0.22 μm filter. Such preparations are described in WO 94/21292. Synthetic derivatives of lipid A are known and thought to be TLR 4 agonists including, but not limited to:
OM174 (2-deoxy-6-O-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026);
OM 294 DP (3S, 9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462);
OM 197 MP-Ac DP (3S-, 9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).

Typically when 3D-MPL is used the antigen and 3D-MPL are delivered with alum or presented in an oil in water emulsion or multiple oil in water emulsions. The incorporation of 3D-MPL is advantageous since it is a stimulator of effector T-cells responses.

Other TLR4 ligands which may be used are alkyl Glucosaminide phosphates (AGPs) such as those disclosed in WO 9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Another immunostimulant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fragments of Quil A have been isolated by HPLC which retain adjuvant activity without the toxicity associated with Quil A (EP 0 362 278), for example QS7 and QS21 (also known as QA7 and QA21). QS21 is a natural saponin derived from the bark of Quillaja saponaria Molina which induces CD8+ cytotoxic T cells (CTLs), Th1 cells and a predominant IgG2a antibody response.

Particular formulations of QS21 have been described which further comprise a sterol (WO 96/33739). The ratio of QS21: sterol will typically be in the order of 1:100 to 1:1 weight to weight. Generally an excess of sterol is present, the ratio of QS21: sterol being at least 1:2 w/w. Typically for human administration QS21 and sterol will be present in a vaccine in the range of about 1 μg to about 100 μg, such as about 10 μg to about 50 μg per dose.

The liposomes generally contain a neutral lipid, for example phosphatidylcholine, which is usually non-crystalline at room temperature, for example eggyolk phosphatidylcholine, dioleoyl phosphatidylcholine or dilauryl phosphatidylcholine. The liposomes may also contain a charged lipid which increases the stability of the lipsome-QS21 structure for liposomes composed of saturated lipids. In these cases the amount of charged lipid is often 1-20% w/w, such as 5-10%. The ratio of sterol to phospholipid is 1-50% (mol/mol), such as 20-25%.

These compositions may contain MPL (3-deacylated mono-phosphoryl lipid A, also known as 3D-MPL). 3D-MPL is known from GB 2 220 211 (Ribi) as a mixture of 3 types of De-O-acylated monophosphoryl lipid A with 4, 5 or 6 acylated chains and is manufactured by Ribi Immunochem, Montana.

The saponins may be separate in the form of micelles, mixed micelles (generally, but not exclusively with bile salts) or may be in the form of ISCOM matrices (EP 0 109 942), liposomes or related colloidal structures such as worm-like or ring-like multimeric complexes or lipidic/layered structures and lamellae when formulated with cholesterol and lipid, or in the form of an oil in water emulsion (for example as in WO 95/17210). Usually, the saponin is presented in the form of a liposomal formulation, ISCOM or an oil in water emulsion.

Immunostimulatory oligonucleotides may also be used. Examples oligonucleotides for use in adjuvants or vaccines of the present invention include CpG containing oligonucleotides, generally containing two or more dinucleotide CpG motifs separated by at least three, more preferably at least six or more nucleotides. A CpG motif is a Cytosine nucleotide followed by a Guanine nucleotide. The CpG oligonucleotides are typically deoxynucleotides. In one embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO 95/26204.

Examples of Oligonucleotides are as Follows:

### TCC ATG ACG TTC CTG ACG TT (CpG 1826)—SEQ ID No. 20

### TCT CCC AGC GTG CGC CAT (CpG 1758)—SEQ ID No. 21

### ACC GAT GAC GTC GCC GGT GAC GGC ACC ACG—SEQ ID No. 22

### TCG TCG TTT TGT CGT TTT GTC GTT (CpG 2006)—SEQ ID No. 23

### TCC ATG ACG TTC CTG ATG CT (CpG 1668)—SEQ ID No. 24

### TCG ACG TTT TCG GCG CGC GCC G (CpG 5456)—SEQ ID No. 25

the sequences may contain phosphorothioate modified internucleotide linkages.

Alternative CpG oligonucleotides may comprise one or more sequences above in that they have inconsequential deletions or additions thereto.

The CpG oligonucleotides may be synthesized by any method known in the art (for example see EP 468520). Conveniently, such oligonucleotides may be synthesized utilising an automated synthesizer.

Examples of a TLR 2 agonist include peptidoglycan or lipoprotein. Imidazoquinolines, such as Imiquimod and Resiquimod are known TLR7 agonists. Single stranded RNA is also a known TLR agonist (TLR8 in humans and TLR7 in mice), whereas double stranded RNA and poly IC (polyinosinic-polycytidylic acid—a commercial synthetic mimetic of viral RNA) are exemplary of TLR 3 agonists. 3D-MPL is an example of a TLR4 agonist whilst CpG is an example of a TLR9 agonist.

An immunostimulant may alternatively or in addition be included. In a one embodiment this immunostimulant will be 3-deacylated monophosphoryl lipid A (3D-MPL).

In one aspect the adjuvant comprises 3D-MPL.

In one aspect the adjuvant comprises QS21.

In one aspect the adjuvant comprises CpG.

In one aspect the adjuvant is formulated as an oil in water emulsion.

In one aspect the adjuvant is formulated as liposomes.

Adjuvants combinations include 3D-MPL and QS21 (EP 0 671 948 B1), oil in water emulsions comprising 3D-MPL and QS21 (WO 95/17210, WO 98/56414), 3D-MPL and QS21 in a liposomal formulation, or 3D-MPL formulated with other carriers (EP 0 689 454 B1). Other adjuvant systems comprise a combination of 3D-MPL, QS21 and a CpG oligonucleotide as described in U.S. Pat. No. 6,558,670 and U.S. Pat. No. 6,544,518.

In one embodiment of the present invention provides a vaccine comprising a stabilized particle as herein described, in combination with 3D-MPL and a diluent. Typically the diluent will be an oil in water emulsion or alum.

Vaccine preparation is generally described in New Trends and Developments in Vaccines, edited by Voller et al., University Park Press, Baltimore, Md., U.S.A., 1978. Encapsulation within liposomes is described, for example, by Fullerton, U.S. Pat. No. 4,235,877.

The amount of the protein particles of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each does will comprise 1-1000 μg of protein, preferably 1-200 μg most preferably 10-100 μg. An optimal amount for a particular vaccine can be ascertained by standard studies involving observation of antibody titres and other responses in subjects. Following an initial vaccination, subjects will preferably receive a boost in about 4 weeks, followed by repeated boosts every six months for as long as a risk of infection exists. The immune response to the protein of this invention is enhanced by the use of adjuvant and or an immunostimulant.

The amount of 3D-MPL used is generally small, but depending on the vaccine formulation may be in the region of 1-1000 μg per dose, for example 1-500 μg per dose, or between 1 to 100 μg per dose, such as 50 or 25 μg per dose.

The amount of CpG or immunostimulatory oligonucleotides in the adjuvants or vaccines of the present invention is generally small, but depending on the vaccine formulation may be in the region of 1-1000 μg per dose, for example 1-500 μg per dose, such as between 1 to 100 μg per dose.

The amount of saponin for use in the adjuvants of the present invention may be in the region of 1-1000 μg per dose, for example 1-500 μg per dose, such as 1-250 μg per dose, particularly between 1 to 100 μg per dose especially 50 or 25 μg per dose.

### Formulations

The formulations of the present invention may be used for both prophylactic and therapeutic purposes. Accordingly the invention provides a vaccine composition as described herein for use in medicine, for example, for the treatment (or phrophylaxis) of malaria (or in the manufacture of a medicament for the treatment/prevention of malaria).

A further aspect of the present invention is to provide a process for the preparation of vaccine components and vaccines and kits comprising elements of the invention, which process comprises expressing DNA sequence encoding the protein, in a suitable host, for example a yeast, and recovering the product as a lipoprotein particle and mixing the latter with at least a stabilizing agent as defined herein, in particular monothioglycerol, cysteine and mixtures thereof, such as monothioglycerol.

The final bulk is usually distributed aseptically in 3 ml glass vials which are then loosely stoppered and transferred to the lyophilizer to undergo a freeze-drying cycle of about 40 h.

In processes for preparing the antigen component the excipients will generally be added and mixed and as the final step the antigen/lipoprotein particle will be added. For this preparation protective measures such as removing oxygen from the vials or protecting the vaccine against light by using amber glass containers may eventually be applied too, in combination with use of a stabilizing agent or as an alternative.

In aspects of the invention wherein the oxygen has removed, the formulation/components/particles etc may be stored under nitrogen.

The adjuvant will often be added to a liquid formulation of the antigen (or a lyophilized formulation of the antigen) to form a vaccine.

A further aspect of the invention lies in a method of treating a patient susceptible to plasmodium infections by administering an effective amount of a vaccine as hereinbefore described.

In a further aspect there is provided an antigenic component for a vaccine or vaccine according to the invention for treatment (or use of same for the manufacture of a medicament for the treatment/prevention of malaria).

The invention also includes prime boost regimes comprising one or more of the various components described herein.

In the context of this specification comprising is to be interpreted as including.

In one aspect the invention provides a stabilized malaria antigen as herein described in a 3 mL glass vial, for example an amber vial, which optionally has been flushed with nitrogen before filling to eliminate oxygen species in vial.

A vial employed may be siliconised or unsiliconised.

The invention also to extends to separate embodiments consisting or consisting essentially of aspects of the invention herein described comprising certain elements, as appropriate.

The examples below are shown to illustrate the methodology, which may be employed to prepare particles of the invention.

### EXAMPLES

### Example 1

Recipe for component for a single pediatric dose of RTS,S malaria vaccine (2 vial formulation)

| Component | Amount | |
|---|---|---|
| RTS,S | 25 | μg |
| NaCl | 2.25 | mg |
| Phosphate buffer (Na/K₂) | 10 | mM |
| Monothioglycerol | 125 | μg |
| Water for Injection | Make volume to 250 | μL |

The above is prepared by adding RTS,S antigen to a mix of Water for Injection, NaCl 1500 mM, phosphate buffer (Na/K₂) 500 mM (pH 6.8 when diluted×50) and an aqueous solution of monothioglycerol at 10%. Finally pH is adjusted to 7.0±0.1.

This may be provided as a vial together with a separate vial of adjuvant, for example a liposomal formulation of MPL and QS21

| Component | Amount | |
|---|---|---|
| 1,2-di-oleoyl-sn-glycero-3-phosphocholine | 500 | μg |
| (DOPC) | | |
| Cholesterol | 125 | μg |
| MPL | 25 | μg |
| QS21 | 25 | μg |
| NaCl | 2.25 | mg |
| Phosphate buffer (Na/K₂) | 10 | mM |
| Water for Injection | Make volume to 250 | μL |

For administration the adjuvant formulation is added to the component formulation, for example using a syringe, and then shaken. Then the dose is administered in the usual way.

The pH of the final liquid formulation is about 6.6+/−0.1.

### Example 1A

A final pediatric liquid formulation (1 vial) according to the invention may be prepared according to the following recipe.

| Component | Amount | |
|---|---|---|
| RTS,S | 25 | μg |
| NaCl | 4.5 | mg |
| Phosphate buffer (Na/K₂) | 10 | mM |
| Monothioglycerol | 125 | μg |
| 1,2-di-oleoyl-sn-glycero-3-phosphocholine | 500 | μg |
| (DOPC) | | |
| Cholesterol | 125 | μg |
| MPL | 25 | μg |
| QS21 | 25 | μg |
| Water for Injection | Make volume to 500 | μL |

The pH of the above liquid formulation is either adjusted to 7.0+/−0.1 (which is favorable for antigen stability, but not favorable at all for the MPL stability), or to 6.1+/−0.1 (which is favorable for MPL stability, but not favorable at all for RTS,S stability). Therefore this formulation is intended for rapid use after preparation.

The above is prepared by adding RTS,S antigen to a mix of Water for Injection, NaC11500 mM, phosphate buffer (Na/K₂) 500 mM (pH 6.8 when diluted×50) and an aqueous solution of monothioglycerol at 10%. Then a premix of liposomes containing MPL with QS21 is added, and finally pH is adjusted.

### Example 1B

A final adult dose (1 vial formulation) for the RTS,S according to the invention may be prepared as follows:

| Component | Amount | |
|---|---|---|
| RTS,S | 50 | μg |
| NaCl | 4.5 | mg |
| Phosphate buffer (Na/K₂) | 10 | mM |
| Monothioglycerol | 250 | μg |
| 1,2-di-oleoyl-sn-glycero-3-phosphocholine | 1000 | μg |
| (DOPC) | | |
| Cholesterol | 250 | μg |
| MPL | 50 | μg |
| QS21 | 50 | μg |
| Water for Injection | Make volume to 500 | μL |

### Example 1C

Example 1C may prepared by putting Example 1, 1A or 1B in an amber vial, for example flushed with nitrogen before filing.

### Example 2

The component according to the invention may also be provided as a bi dose for use in pediatric population (2 vial formulation).

| Component | Amount | |
|---|---|---|
| RTS,S | 50 | μg |
| NaCl | 4.5 | mg |
| Phosphate buffer (Na/K₂) | 10 | mM |
| Monothioglycerol | 250 | μg |
| Water for Injection | Make volume to 500 | μL |

The above is prepared by adding RTS,S antigen to a mix of Water for Injection, NaCl 1500 mM, phosphate buffer (Na/K₂) 500 mM (pH 6.8 when diluted×50) and an aqueous solution of monothioglycerol at 10%. Finally pH is adjusted to 7.0±0.1.

This may be provided a vial together with a separate vial of adjuvant, for example a liposomal formulation of MPL and QS21

| Component | Amount | |
|---|---|---|
| 1,2-di-oleoyl-sn-glycero-3-phosphocholine | 1000 | μg |
| (DOPC) | | |
| Cholesterol | 250 | μg |
| MPL | 50 | μg |
| QS21 | 50 | μg |
| NaCl | 4.5 | mg |
| Phosphate buffer (Na/K₂) | 10 | mM |
| Water for Injection | Make volume to 500 | μL |

For administration the adjuvant formulation is added to the component formulation, for example using a syringe, and then shaken. Then a single dose is withdrawn (500 μL) and is administered in the usual way.

The pH of the final liquid formulation is about 6.6+/−0.1.

### Example 2A

A final pediatric liquid formulation (1 vial) according to the invention may be prepared as a bidose according to the following recipe.

| Component | Amount | |
|---|---|---|
| RTS,S | 50 | μg |
| NaCl | 9 | mg |
| Phosphate buffer (Na/K₂) | 10 | mM |
| Monothioglycerol | 250 | μg |
| 1,2-di-oleoyl-sn-glycero-3-phosphocholine | 1000 | μg |
| (DOPC) | | |
| Cholesterol | 250 | μg |
| MPL | 50 | μg |
| QS21 | 50 | μg |
| Water for Injection | Make volume to 1000 | μL |

The pH of the above liquid formulation is either adjusted to 7.0+/−0.1 (which is favorable for antigen stability, but not favorable at all for the MPL stability), or to 6.1+/−0.1 (which is favorable for MPL stability, but not favorable at all for RTS,S stability). Therefore this formulation is intended for rapid use after preparation.

The above is prepared by adding RTS,S antigen to a mix of Water for Injection, NaC11500 mM, phosphate buffer (Na/K₂) 500 mM (pH 6.8 when diluted×50) and an aqueous solution of monothioglycerol at 10%. Then a premix of liposomes containing MPL with QS21 is added, and finally pH is adjusted.

### Example 2B

A final adult dose (1 vial formulation) for the RTS,S according to the invention may be prepared as a bidose as follows:

| Component | Amount | |
|---|---|---|
| RTS,S | 100 | μg |
| NaCl | 9 | mg |
| Phosphate buffer (Na/K₂) | 10 | mM |
| Monothioglycerol | 500 | μg |
| 1,2-di-oleoyl-sn-glycero-3-phosphocholine | 2000 | μg |
| (DOPC) | | |
| Cholesterol | 500 | μg |
| MPL | 100 | μg |
| QS21 | 100 | μg |
| Water for Injection | Make volume to 1000 | μL |

### Example 2C

Example 2C may prepared by putting Example 2, 2A or 2B in an amber vial, for example flushed with nitrogen before filling.

### Example 3

Recipe for component for a single pediatric dose of RTS,S malaria vaccine (2 vial formulation) with a filling volume of 500 μl

| Component | Amount |
|---|---|
| RTS,S | 25 μg |
| NaCl | 4.5 mg |
| Phosphate buffer (Na/K₂) | 10 mM |
| Monothioglycerol | 50 μg or 200 μg |
| Water for Injection | Make volume to 500 μL |

The above is prepared by adding RTS,S antigen to a mix of Water for Injection, NaC11500 mM, phosphate buffer (Na/K₂) 500 mM (pH 6.8 when diluted×50) and an aqueous solution of monothioglycerol at 10%. Finally pH is adjusted to 7.0±0.1.

This may be provided as a vial together with a separate vial of adjuvant, for example a liposomal formulation of MPL and QS21 with a filling volume of 500 μl.

| Component | Amount | |
|---|---|---|
| 1,2-di-oleoyl-sn-glycero-3-phosphocholine | 500 | μg |
| (DOPC) | | |
| Cholesterol | 125 | μg |
| MPL | 25 | μg |
| QS21 | 25 | μg |
| NaCl | 4.5 | mg |
| Phosphate buffer (Na/K₂) | 10 | mM |
| Water for Injection | Make volume to 500 | μL |

For administration the adjuvant formulation is added to the component formulation, for example using a syringe, and then shaken. Then the dose is administered in the usual way.

The pH of the final liquid formulation is about 6.6+/−0.1 and the injection volume is 1 ml.

### Example 4

Accelerated stability results indicate the following:
pH and osmolality are compatible with injection;
in terms of RTS,S content: after 5 weeks at 4° C. or 37° C., there is no antigen loss by non-specific adsorption;
with respect to antigen integrity (see FIGS. 6, 7 and 8):
   no significant degradation after accelerated stability (7 days 37° C.±AOT, 14 days 37° C.);
   without inerting, an antioxidant (monothioglycerol) is required to avoid oxidative aggregation after accelerated stability (7d 37° C.±AOT, 14d 37° C.):
      0.01% sufficient to avoid aggregation at 37° C.;
      0.04% required for stability at 37° C.+AOT;
   amber glass ensures antigen protection against light (as seen after AOT);
   no modification of RTS,S particle size distribution after accelerated stability (7 days at 37° C.);
with respect to antigenicity (see FIG. 9):
   without inerting, an antioxidant (monothioglycerol) is required to avoid oxidative aggregation and antigenicity increase after accelerated stability (7 days at 37° C.±AOT):
      0.01% allows a very stable antigenicity (80-120%);
      0.04% induces a slight antigenicity decrease (−10%) after accelerated stability;
upon mixing with AS01 (liposomal adjuvant formulation with MPL and QS21):
   RTS,S integrity and antigenicity are maintained for at least 24 hours at 25° C. post-mixing.

SDS-Page analyses have been performed after storage of these RTS,S liquid formulations with or without monothioglycerol, for 7 days, 14 days, FIG. 8or even 5 weeks at 37° C.

FIG. 6 shows
in absence of monothioglycerol: slight RTS,S aggregation after 7 days storage at 37° C. (wells 3 and 4); complete aggregation and slight degradation in white vials stored for 7 days at 37° C. before exposure to AOT (well 6), while amber glass protects RTS,S against degradation and oxidative aggregation induced by light (well 5);
in presence of monothioglycerol 0.01%: this concentration is sufficient to stabilize RTS,S for 7 days storage at 37° C. (well 7), but not when an Accelerated Oxidation Test (AOT) is cumulated (well 9), excepted when combined with amber glass (well 8);
in presence of monothioglycerol 0.04%: this concentration is sufficient to stabilize RTS,S for 7 days storage at 37° C. (well 10), also when cumulated with an Accelerated Oxidation Test (well 12); in this case filling in amber glass vials (well 11) is not required;
that mixing with AS01 has no impact on RTS,S profile, even after 24 h storage at 25° C.

FIG. 7 shows that monothioglycerol is required to avoid RTS,S aggregation, but both concentrations are able to stabilize RTS,S for at least 14 days storage at 37° C. (wells 11 and 12 vs. well 10).

FIG. 8 shows that after 5 weeks at 37° C. RTS,S is aggregated and degraded in all formulations; AS01 worsens aggregation in all formulations.

### Example 5

RTS,S antigenicity was determined by mixed ELISA αCSP-αS on formulations containing 0, 0.01 or 0.04% monothioglycerol, at T0 (±AOT) or after 7d (±AOT) or 5 w storage at 37° C.; it has been measured before, just after and 24 h 25° C. post-reconstitution with AS01.

FIG. 9 shows
in absence of monothioglycerol:
   exposure to 675 W for 15 h (AOT) provokes an increase in antigenicity of 50-60% (this may be linked to oxidative aggregation observed in SDS-Page), but filling in amber glass vials limits this antigenicity increase to ˜20%;
   storage for 7 days at 37° C. provokes an increase in antigenicity of ˜30-40% (this also may be linked to oxidative aggregation observed in SDS-Page);
   antigenicity decrease of ˜30% between 7 days and 5 weeks 37° C.;
   after 24 h at 25° C. AS01 provokes an increase in antigenicity of ˜20% (this also may be linked to increase in aggregation observed in SDS-Page);
in presence of 0.01% monothioglycerol:
   monothioglycerol protects RTS,S against antigenicity increase induced by storage for 7 days at 37° C., AOT (rendering amber glass useless) or mixing with AS01 (but increase of ˜20% when storage for 24 h at 25° C. in AS01 is cumulated to 7d storage at 37° C.);
   antigenicity decrease of ˜20% between 7d and 5 w 37° C. (→out of spec);
in presence of 0.04% monothioglycerol:
   monothioglycerol protects RTS,S against antigenicity increase induced by AOT, rendering amber glass useless;
   storage for 7 days at 37° C. provokes a decrease in antigenicity of ˜20%;
   no antigenicity decrease between 7d and 5 w 37° C.;
   after 24 h at 25° C. AS01 provokes an increase in antigenicity of ˜30-40%.

### Example 6

To investigate the impact of monothioglycerol fixation to RTS,S on recognition of RF1-epitope (S) by monoclonal antibodies, the reactivity of RTS,S for RF1 ascitic fluid by ELISA inhibition assay has been determined in RTS,S liquid formulations, stabilized or not by monothioglycerol (MTG), at T0 (time 0) or after 7d (7 days) storage at 37° C. (see Table 1).

TABLE 1
Reactivity of RTS,S lots for RF1 ascitic fluid by Elisa inhibition
assay.

| | | | |
|---|---|---|---|
| RTSS lyo (100 μg/ml) | 5612 | 7975 | 6794 |
| Purified bulk (ERTSAPA001) T0 | 7890 | 6783 | 7337 |
| Purified bulk (ERTSAPA001) 7 d 37° C. | 1393 | 993 | 1193 |
| RTS,S in NaCl PO4 T0 | 4117 | 3528 | 3823 |
| RTS,S in NaCl PO4 7 d 37° C. | 524 | 544 | 534 |
| RTS,S + MTG 0.02% T0 | 5732 | 5099 | 5416 |
| RTS,S + MTG 0.02% 7 d 37° C. | 4310 | 4379 | 4345 |
| RTS,S + MTG 0.08% T0 | 5285 | 6438 | 5862 |
| RTS,S + MTG 0.08% 7 d 37° C. | 4401 | 4268 | 4335 |

Monclonal antibodies to epitope RF1 were employed in the assay shown in Table 1.

In

Table 1 only 2 samples have RF1-epitopes that are significantly better recognized than the others:
RTS,S Purified Bulk stored for 7 days at 37° C.;
RTS,S in liquid formulation containing no monothioglycerol and stored for 7 days at 37° C.

This means that a conformational change occurs in these samples, increasing RF1-epitope accessibility. These results have to be considered in parallel with results of mixed ELISA αCPS-αS (increase in RTS,S antigenicity in formulation without monothioglycerol after storage for 7 days at 37° C.).

Therefore we may conclude
that monothioglycerol seems to have no negative impact on recognition/accessibility of RF1-epitope at T0 (same level as in “fresh” purified bulk);
that level of recognition remains stable in RTS,S liquid formulations containing monothioglycerol and stored for 7d at 37° C., indicating that RTS,S conformation is stabilized by monothioglycerol.

### Immunogenicity Data

### Example 7

The immunogenicity of several RTS,S formulations was evaluated and compared in mice. In these experiments, the RTS,S, AS01, 50 mM PO₄, NaCl 100 mM, pH 6.1 vaccine formulation was used as a benchmark for the evaluation of 3 other RTS,S formulations, i.e.

1) the mannitol-sucrose lyophilized RTS,S (to be reconstituted with adjuvant),
2) the liquid formulation containing 0.02% monothioglycerol and
3) the liquid formulation containing 0.08% monothioglycerol (each to be mixed with AS01 before injection).

Of note, after mixing of liquid RTS,S formulations with AS01, the final concentrations of monothioglycerol were 0.01% and 0.04%.

The humoral and cellular immune responses elicited by the different RTS,S formulations were determined in two different types of immunogenicity experiments described below and in Example 8 respectively.

Mouse Humoral Immune Response Experiments

a. Introduction

The anti-CS and anti-HBs antibody responses (total immunoglobulins) elicited in mice immunized with the different RTS,S formulations were evaluated and compared.

b. Experimental Design

The experimental design followed the one from the current in vivo potency assay of the RTS,S/AS01 vaccine, i.e. the Balb/C mouse strain, a single intra-peritoneal injection of the dose release from the in vivo potency assay (0.25 μg RTS,S) and the measurement by ELISA of the anti-CS & anti-HBs antibody responses (total immunoglobulins) in the sera at 28 days post-immunization.

In order to define the sample size of the experiment, the variability of the anti-CS and anti-HBs antibody responses estimated from the in vivo potency assay performed with RTS, S formulated with AS01 adjuvant, was used. Based on this, the statisticians determined that a sample size of 25 mice per group (in 2 different experiments) would allow the detection of a 2-fold difference between the group means in a two-way ANOVA with a power of 90.9%.

c. Results

The anti-CS serology (total Ig) was performed using the sera collected 28 days post-immunization. The titres from the 50 mice/group were expressed in Log and are presented in FIG. 10.

The statistical analysis (Dunnett) associated with the anti-CS serology results is summarized in Table 4.

TABLE 4
Statistical comparisons of anti-CS GMTs between
various RTS,S formulations

| Group_1 | Group_2 | Adjustment | Prob_Adj |
|---|---|---|---|
| RTS,S mannitol- | RTS,S sucrose | Dunnett | 0.2529 |
| sucrose lyo | lyo | | |
| RTS,S Liq 0.02% | RTS,S sucrose | Dunnett | 0.6070 |
| monothioglycerol | lyo | | |
| RTS,S Liq 0.08% | RTS,S sucrose | Dunnett | 0.6025 |
| monothioglycerol | lyo | | |

These results indicate that the anti-CS total Ig response elicited by the mannitol-sucrose lyo or the liquid RTS,S formulations were not statistically different from the one induced by RTS,S when formulated in a liposomal adjuvant formulation of MPL and QS21 (statistical power of 92.7% to detect a 2 fold difference in Ab titres).

The anti-HBs serology (total Ig) was performed using the sera collected 28 days post-immunization. The titres from the 50 mice/group were expressed in Log and are presented in FIG. 11.

The statistical analysis (Dunnett) associated with the anti-HBs serology results is summarized in Table 5.

TABLE 5
Statistical comparisons of anti-HBs GMTs between individual
alternative RTS,S formulations and the current RTS,S formulation

| Group_1 | Group_2 | Adjustment | Prob_Adj |
|---|---|---|---|
| RTS,S mannitol- | RTS,S sucrose | Dunnett | 0.1332 |
| sucrose lyo | yo | | |
| RTS,S Liq 0.02% | RTS,S sucrose | Dunnett | 0.9857 |
| monothioglycerol | lyo | | |
| RTS,S Liq 0.08% | RTS,S sucrose | Dunnett | 0.1105 |
| monothioglycerol | lyo | | |

These results indicate that the anti-HBs total Ig response elicited by the mannitol-sucrose lyo or the liquid RTS,S formulations were not statistically different from the one induced by RTS,S when formulated in a liposomal formulation of MPL and QS21 (statistical power of 91.4% to detect a 2 fold difference in Ab titres).

d. Conclusions

All three alternatives RTS,S formulations tested elicited anti-CS and anti-HBs antibody responses in mice. In addition, the statistical analysis indicated that the anti-CS and anti-HBs antibody responses elicited by either the mannitol-sucrose RTS,S lyo, liquid RTS,S 0.02% monothioglycerol or liquid RTS,S 0.08% monothioglycerol reconstituted extemporaneously in a liposomal formulation of MPL and QS21, were not statistically significantly different from the ones induced by the current RTS,S lyophilized formulation reconstituted in AS01. The power associated to the analysis of anti-CS and anti-HBs antibody responses were respectively at least 92.7% and 91.4% to show ratio of 2 (original scale, i.e. antibody titres) or differences of 0.301 (log scale).

### Example 8

### Mouse Cellular Immune Response Experiments

a. Introduction

In this second type of experiment, cell mediated immune (CMI) responses to HBs and CS antigens were measured using flow cytometry-based detection of cytokine expressing T cells following short term ex vivo stimulation with pools of peptides covering the HBs and CS sequences.

b. Experimental Design

The groups tested are the same as the groups from the experiments described above in Example 7. However, the experimental design was different, i.e. C57BL/6 mice were immunized 3 times intramuscularly with a dose range (5 μg and 2.5 μg) of RTS,S antigen in AS01, in accordance with protocols from previous mouse immunogenicity studies aimed at assessing antigen-specific cellular immune responses. The experiment was performed twice and the sample size was determined in order to collect enough cells to perform the flow cytometry-based assay. Indeed, in each group, CMI analysis was performed on blood cells pooled from 4 mice (i.e. 3 pools/group). This read-out is considered as exploratory because no statistical conclusion can be drawn with only three values (pools) available per group per experiment and because of the well known variability of such cell-based assays.

c. Results

The CS-specific and HBs-specific CD4 and CD8 T cell responses at 7 days post 3^{rd} immunization are presented in FIGS. 12, 13, 14 and 15.

Each triangle within each graph (i) represents the response from a pool of 4 mice after in vitro restimulation of the peripheral blood lymphocytes with peptide pools covering the CS or HBs sequences and (ii) represents the percentage of CD4 or CD8 T cells producing IL-2 and/or IFN-gamma in response to the peptide pools used in the in vitro restimulation.

These results indicate that CS- and HBs-specific CD4 T cell responses are elicited by all RTS,S formulations tested. These antigen-specific CD4 T cell responses are comparable whether RTS,S & AS01 (current lyophilized formulation), RTS,S mannitol-sucrose lyo, liquid RTS,S containing 0.02% or 0.08% monothioglycerol (MTG) are used for the immunization (responses comparable at all dose tested).

These results indicate that CS- and HBs-specific CD8 T cell responses are elicited by all RTS,S formulations tested. These antigen-specific CD8 T cell responses are comparable whether RTS,S & AS01 (current lyophilized formulation), RTS,S mannitol-sucrose lyo, liquid RTS,S containing 0.02% or 0.08% monothioglycerol (MTG) are used for the immunization (responses comparable at all dose tested). Of note, there is a tendency for liquid RTS,S formulations to induce higher percentages of Ag-specific CD8 T cells at both doses tested (2.5 & 5 μg). However, as mentioned above, this read-out was considered as exploratory because no statistical conclusion can be drawn with only three values (pools) available per group per experiment and because of the well known variability of such cell-based assays.

d. Conclusions

The CS- and HBs-specific CD4 and CD8 T cell responses elicited by RTS,S mannitol-sucrose lyo, liquid RTS,S 0.02% monothioglycerol and liquid RTS,S 0.08% monothioglycerol are comparable to the ones elicited by the current RTS,S lyophilized formulation when reconstituted in AS01.

### REFERENCE

(1) Harford N, Cabezon T, Colau B, et al., “Construction and Characterization of a Saccharomyces Cerevisiae Strain (RIT4376) Expressing Hepatitis B Surface Antigen”, Postgrad Med J 63, Supp. 2: 65-70, 1987.
(2) Jacobs E, Rutgers T, Voet P, et al., “Simultaneous Synthesis and Assembly of Various Hepatitis B Surface Proteins in Saccharomyces cerevisiae”, Gene 80: 279-291, 1989.
(3) Vieira J and Messing J, “The pUC plasmids, an M13 mp 7-Derived System for Insertion Mutagenesis and Sequencing with Synthetic Universal Primers”, Gene 19: 259-268, 1982.
(4) Hinnen A, Hicks J B, and Fink G R, “Transformation of Yeast”, Proc Natl Acad Sci USA 75: 1929-1933, 1980.
(5) Broach J R, Strathern J N, and Hicks J B, “Transformation in Yeast Development of a Hybrid Cloning Vector and Isolation of the CAN 1 Gene”, Gene 8: 121-133, 1979.
(6) Zhang H, et al., “Double Stranded SDNA Sequencing as a Choice for DNA Sequencing”, Nucleic Acids Research 16: 1220, 1988.
(7) Dame J B, Williams J L. Mc Cutchan T F, et al., “Structure of the Gene Encoding the Immunodominant Surface Antigen on the Sporozoites of the Human Malaria Parasite Plasmodium falciparum”, Science 225: 593-599, 1984.
(8) Valenzuela P, Gray P, Quiroga M, et al., “Nucleotide Sequences of the Gene Coding for the Major Protein of Hepatitis B Virus Surface Antigen”, Nature 280: 815-819, 1979.
(9) In S S, Kee-Hoyung L, Young R K, et al., “comparison of Immunological Responses to Various Types of Circumsporozoite Proteins of Plasmodium vivax in Malaria Patients of Korea”, Microbiol. Immunol. 48(2): 119-123, 2004; Microbiol. Immunol. 2004; 48(2): 119-123.
(10) Rathore D, Sacci J B, de la Vega P, et al., “Binding and Invasion of Liver Cells by Plasmodium falciparum Sporozoites”, J. Biol. Chem. 277(9): 7092-7098, 2002. Rathore et al., 2002, J. Biol. Chem. 277, 7092-8.

SEQUENCE LISTING

SEQ ID NO: 1
REGION 1
KLKQP
SEQ ID NO: 2
REGION II PLUS
CSVTCG
SEQ ID NO: 3
VK210 repeat
GDRAAGQPA
SEQ ID NO: 4
VK210 repeat
GDRADGQPA
SEQ ID NO: 5
VK210 repeat
GDRADGQAA
SEQ ID NO: 6
VK210 repeat
GNGAGGQPA
SEQ ID NO: 7
VK210 repeat
GDGAAGQPA
SEQ ID NO: 8
VK210 repeat
GDRAA GQAA
SEQ ID NO: 9
VK210 repeat
GNGAGGQAA
SEQ ID NO: 10
Major VK247 repeat
ANGAGNQPG
SEQ ID NO: 11
12 amino acid insert
GGNAANKKAEDA
SEQ ID NO: 12
Pv-CS nucleotide sequence
Acacattgcggacataatgtagatttatctaaagctataaatttaaatggtgtaaacttc
aataacgtagacgctagttcactcggggctgcg
cacgtaggtcagtctgctagcagggggcgcggtctcggggaaaacccagacgacgaagaa
ggtgatgctaaaaagaaaaaggacg
gtaaaaaagcggaaccaaaaaatccaagggaaaataaattaaaacagcccggggatcgcg
cggatggtcaagcggcgggtaatggg
gcggggggtcaaccagcgggggatcgcgcggctggtcagccagcgggggatcgcgcggct
ggtcagccagcgggggatggtgc
ggctggccaaccagcgggggatcgcgcggatggtcagccagcgggggatcgcgcggatgg
tcaaccagccggtgatcgcgcggct
ggccaagcggccggtaatggggcggggggtcaagcggccgcgaacggagcggggaaccag
ccaggcggcggtaacgctgcga
ataaaaaagcggaagatgcgggtggtaacgcgggcggtaatgcgggcggccaaggtcaga
acaacgaaggggctaatgcaccaaa
cgaaaaatctgtcaaagaatatctcgataaagtccgcgctacagtagggacagaatggac
gccatgctctgtaacatgtggtgtcggggt
acgcgtgcgccgccgtgtcaatgcggctaacaaaaaaccagaagatctcacgttaaatga
tctcgaaacggatgtctgcaca
SEQ ID NO: 13
Amino acid sequence of Pv-CS protein
THCGHNVDLSKAINLNGVNFNNVDASSLGAAHVGQSASRGRGLGEN
PDDEEGDAKKKKDGKKAEPKNPRENKLKQPGDRADGQAAGNGAGG
QPAGDRAAGQPAGDRAAGQPAGDGAAGQPAGDRADGQPAGDRADG
QPAGDRAAGQAAGNGAGGQAAANGAGNQPGGGNAANKKAEDAGG
NAGGNAGGQGQNNEGANAPNEKSVKEYLDKVRATVGTEWTPCSVT
CGVGVRVRRRVNAANKKPEDLTLNDLETDVCT
SEQ ID NO: 14
Minor Type 2 repeat
ANGAGDQPG
SEQ ID No 15
CSV HYBRID GENE

| | |
|---|---|
| ACCCATTGTGGTCACAATGTCGATTTGTCTAAGGCCATTAACTTGAACGGTGTTAATTTC | 60 |
| AACAACGTCGATGCTTCTTCTTTAGGTGCCGCTCATGTTGGTCAATCTGCTTCAAGAGGT | 120 |
| AGAGGTTTAGGTGAAAACCCAGACGACGAAGAAGGTGACGCTAAGAAGAAGAAGGACGGT | 180 |
| AAGAAGGCCGAACCAAAGAACCCAAGAGAAAACAAGTTGAAACAACCAGGTGACAGAGCC | 240 |
| GACGGACAAGCAGCTGGTAATGGTGCTGGAGGTCAACCAGCTGGTGACAGAGCTGCCGGT | 300 |
| CAGCCTGCTGGTGATAGAGCTGCTGGACAACCTGCTGGAGACGGTGCCGCCGGTCAACCT | 360 |
| GCTGGTGATAGAGCAGACGGACAACCAGCTGGTGACCGTGCTGACGGACAGCCAGCCGGC | 420 |
| GATAGGGCTGCAGGTCAAGCCGCTGGTAACGGTGCCGGTGGTCAAGCTGCTGCTAACGGT | 480 |
| GCTGGTAACCAACCAGGTGGTGGTAACGCTGCCAACAAGAAAGCTGAAGACGCTGGTGGT | 540 |
| AATGCTGGAGGTAATGCAGGTGGTCAGGGTCAAAACAACGAAGGTGCTAACGCTCCAAAC | 600 |
| GAAAAGTCTGTTAAGGAATACTTAGATAAGGTTAGAGCTACTGTCGGTACTGAATGGACT | 660 |
| CCATGTTCTGTTACTTGTGGTGTCGGTGTTAGAGTTAGAAGAAGAGTTAACGCCGCTAAC | 720 |
| AAGAAGCCAGAAGACTTGACTCTAAACGACTTGGAAACTGACGTTTGTACT | 771 |
| SEQ ID No 16 | |
| CSV-S fusion | |
| Nucleotide sequence | |
| ATGATGGCTCCCGGGACCCATTGTGGTCACAATGTCGATTTGTCTAAGGCCATTAACTTG | 60 |
| AACGGTGTTAATTTCAACAACGTCGATGCTTCTTCTTTAGGTGCCGCTCATGTTGGTCAA | 120 |
| TCTGCTTCAAGAGGTAGAGGTTTAGGTGAAAACCCAGACGACGAAGAAGGTGACGCTAAG | 180 |
| AAGAAGAAGGACGGTAAGAAGGCCGAACCAAAGAACCCAAGAGAAAACAAGTTGAAACAA | 240 |
| CCAGGTGACAGAGCCGACGGACAAGCAGCTGGTAATGGTGCTGGAGGTCAACCAGCTGGT | 300 |
| GACAGAGCTGCCGGTCAGCCTGCTGGTGATAGAGCTGCTGGACAACCTGCTGGAGACGGT | 360 |
| GCCGCCGGTCAACCTGCTGGTGATAGAGCAGACGGACAACCAGCTGGTGACCGTGCTGAC | 420 |
| GGACAGCCAGCCGGCGATAGGGCTGCAGGTCAAGCCGCTGGTAACGGTGCCGGTGGTCAA | 480 |
| GCTGCTGCTAACGGTGCTGGTAACCAACCAGGTGGTGGTAACGCTGCCAACAAGAAAGCT | 540 |
| GAAGACGCTGGTGGTAATGCTGGAGGTAATGCAGGTGGTCAGGGTCAAAACAACGAAGGT | 600 |
| GCTAACGCTCCAAACGAAAAGTCTGTTAAGGAATACTTAGATAAGGTTAGAGCTACTGTC | 660 |
| GGTACTGAATGGACTCCATGTTCTGTTACTTGTGGTGTCGGTGTTAGAGTTAGAAGAAGA | 720 |
| GTTAACGCCGCTAACAAGAAGCCAGAAGACTTGACTCTAAACGACTTGGAAACTGACGTT | 780 |
| TGTACTCCCGGGCCTGTGACGAACATGGAGAACATCACATCAGGATTCCTAGGACCCCTG | 840 |
| CTCGTGTTACAGGCGGGGTTTTTCTTGTTGACAAGAATCCTCACAATACCGCAGAGTCTA | 900 |
| GACTCGTGGTGGACTTCTCTCAATTTTCTAGGGGGATCACCCGTGTGTCTTGGCCAAAAT | 960 |
| TCGCAGTCCCCAACCTCCAATCACTCACCAACCTCCTGTCCTCCAATTTGTCCTGGTTAT | 1020 |
| CGCTGGATGTGTCTGCGGCGTTTTATCATATTCCTCTTCATCCTGCTGCTATGCCTCATC | 1080 |
| TTCTTATTGGTTCTTCTGGATTATCAAGGTATGTTGCCCGTTTGTCCTCTAATTCCAGGA | 1140 |
| TCAACAACAACCAATACGGGACCATGCAAAACCTGCACGACTCCTGCTCAAGGCAACTCT | 1200 |
| ATGTTTCCCTCATGTTGCTGTACAAAACCTACGGATGGAAATTGCACCTGTATTCCCATC | 1260 |
| CCATCGTCCTGGGCTTTCGCAAAATACCTATGGGAGTGGGCCTCAGTCCGTTTCTCTTGG | 1320 |
| CTCAGTTTACTAGTGCCATTTGTTCAGTGGTTCGTAGGGCTTTCCCCCACTGTTTGGCTT | 1380 |
| TCAGCTATATGGATGATGTGGTATTGGGGGCCAAGTCTGTACAGCATCGTGAGTCCCTTT | 1440 |
| ATACCGCTGTTACCAATTTTCTTTTGTCTCTGGGTATACATTTAA | 1485 |

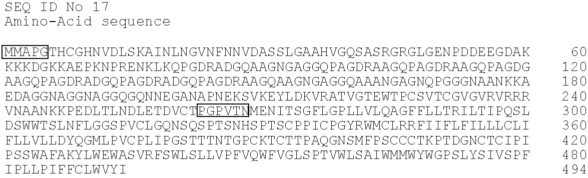

SEQ ID NOs. 18 and 19
Nucelotide sequence of the RTS expression cassette and predicted
translation product of the RTS-HBsAg hybrid protein.
The translation product initiated from the TDH3 ATG codon is shown
below the DNA sequence.
AAGCTTACCAGTTCTCACACGGAACACCACTAATGGACACAAATTCGAAATACTTTGACC
CTATTTTCGAGGACCTTGTCACCTTGAGCCCAAGAGAGCCAAGATTTAAATTTTCCTATG
ACTTGATGCAAATTCCCAAAGCTAATAACATGCAAGACACGTACGGTCAAGAAGACATAT
TTGACCTCTTAACTGGTTCAGACGCGACTGCCTCATCAGTAAGACCCGTTGAAAAGAACT
TACCTGAAAAAAACGAATATATACTAGCGTTGAATGTTAGCGTCAACAACAAGAAGTTTA
ATGACGCGGAGGCCAAGGCAAAAAGATTCCTTGATTACGTAAGGGAGTTAGAATCATTTT
GAATAAAAAACACGCTTTTTCAGTTCGAGTTTATCATTATCAATACTGCCATTTCAAAGA
ATACGTAAATAATTAATAGTAGTGATTTTCCTAACTTTATTTAGTCAAAAATTAGCCTTT
TAATTCTGCTGTAACCCGTACATGCCCAAAATAGGGGGCGGGTTACACAGAATATATAAC
ATCGTAGGTGTCTGGGTGAACAGTTTATCCCTGGCATCCACTAAATATAATGGAGCTCGC
TTTTAAGCTGGCATCCAGAAAAAAAAAGAATCCCAGCACCAAAATATTGTTTTCTTCACC
AACCATCAGTTCATAGGTCCATTCTCTTAGCGCAACTACAGAGAACAGGGGCACAAACAG
GCAAAAAACGGGCACAACCTCAATGGAGTGATGCAACCTGCCTGGAGTAAATGATGACAC
AAGGCAATTGACCCACGCATGTATCTATCTCATTTTCTTACACCTTCTATTACCTTCTGC
TCTCTCTGATTTGGAAAAAGCTGAAAAAAAAGGTTGAAACCAGTTCCCTGAAATTATTCC
CCTACTTGACTAATAAGTATATAAAGACGGTAGGTATTGATTGTAATTCTGTAAATCTGTAAATCTAT
TTCTTAAACTTCTTAAATTCTACTTTTATAGTTAGTCTTTTTTTTAGTTTTAAAACACCA
AGAACTTAGTTTCGAATAAACACACATAAACAAACAAAATGATGGCTCCCGATCCTAATG
MetMetAlaProAspProAsnA
CAAATCCAAATGCAAACCCAAATGCAAACCCAAACGCAAACCCCAATGCAAATCCTAATG
LaAsnProAsnAlaAsnProAsnAlaAsnProAsnAlaAsnProAsnAlaAsnProAsnA
CAAACCCCAATGCAAATCCTAATGCAAATCCTAATGCCAATCCAAATGCAAATCCAAATG
LaAsnProAsnAlaAsnProAsnAlaAsnProAsnAlaAsnProAsnAlaAsnProAsnA
CAAACCCAAACGCAAACCCCAATGCAAATCCTAATGCCAATCCAAATGCAAATCCAAATG
LaAsnProAsnAlaAsnProAsnAlaAsnProAsnAlaAsnProAsnAlaAsnProAsna
CAAACCCAAATGCAAACCCAAATGCAAACCCCAATGCAAATCCTAATAAAAACAATCAAG
LaAsnProAsnAlaAsnProAsnAlaAsnProAsnAlaAsnProAsnLysAsnAsnGlnG
GTAATGGACAAGGTCACAATATGCCAAATGACCCAAACCGAAATGTAGATGAAAATGCTA
LyAsnGlyGlnGlyHisAsnMetProAsnAspProAsnAspProAsnArgAsnValAspGluAsnAlaA
ATGCCAACAATGCTGTAAAAAATAATAATAACGAAGAACCAAGTGATAAGCACATAGAAC
snAlaAsnAsnAlaValLysAsnAsnAsnAsnGluGluProSerAspLysHisIleGluG
AATATTTAAAGAAAATAAAAAATTCTATTTCAACTGAATGGTCCCCATGTAGTGTAACTT
LnTyrLeuLysLysIleLysAsnSerIleSerThrGluTrpSerProCysSerValThrC
GTGGAAATGGTATTCAAGTTAGAATAAAGCCTGGCTCTGCTAATAAACCTAAAGACGAAT
YsGlyAsnGlyIleGlnValArgIleLysProGlySerAlaAsnLysProLysAspGluL
TAGATTATGAAAATGATATTGAAAAAAAAATTTGTAAAATGGAAAAGTGCTCGAGTGTGT
euAspTyrGluAsnAspIleGluLysLysIleCysLysMetGluLysCysSerSerValP
TTAATGTCGTAAATAGTCGACCTGTGACGAACATGGAGAACATCACATCAGGATTCCTAG
HeAsnValValAsnSerArgProValThrAsnMetGluAsnIleThrSerGlyPheLeuG
GACCCCTGCTCGTGTTACAGGCGGGGTTTTTCTTGTTGACAAGAATCCTCACAATACCGC
LyProLeuLeuValLeuGlnAlaGlyPhePheLeuLeuThrArgIleLeuThrIleProG
AGAGTCTAGACTCGTGGTGGACTTCTCTCAATTTTCTAGGGGGATCACCCGTGTGTCTTG
LnSerLeuAspSerTrpTrpThrSerLeuAsnPheLeuGlyGlySerProValCysLeuG
GCCAAAATTCGCAGTCCCCAACCTCCAATCACTCACCAACCTCCTGTCCTCCAATTTGTC
LyGlnAsnSerGlnSerProThrSerAsnHisSerProThrSerCysProProIleCysP
CTGGTTATCGCTGGATGTGTCTGCGCGTTTTATCATATTCCTCTTCATCCTGCTGCTAT
RoGlyTyrArgTrpMetCysLeuArgArgPheIleIlePheLeuPheIleLeuLeuLeuC
GCCTCATCTTCTTATTGGTTCTTCTGGATTATCAAGGTATGTTGCCCGTTTGTCCTCTAA
YsLeuIlePheLeuLeuValLeuLeuAspTyrGlnGlyMetLeuProValCysProLeul
TTCCAGGATCAACAACAACCAATACGGGACCATGCAAAACCTGCACGACTCCTGCTCAAG
LeProGlySerThrThrThrAsnThrGlyProCysLysThrCysThrThrProAlaGlnG
GCAACTCTATGTTTCCCTCATGTTGCTGTACAAAACCTACGGATGGAAATTGCACCTGTA
LyAsnSerMetPheProSerCysCysCysThrLysProThrAspGlyAsnCysThrCysl
TTCCCATCCCATCGTCCTGGGCTTTCGCAAAATACCTATGGGAGTGGGCCTCAGTCCGTT
LeProIleProSerSerTrpAlaPheAlaLysTryLeuTrpGluTrpAlaSerValArgP
TCTCTTGGCTCAGTTTACTAGTGCCATTTGTTCAGTGGTTCGTAGGGCTTTCCCCCACTG
HeSerTrpLeuSerLeuLeuValProPheValGlnTrpPheValGlyLeuSerProThrV
TTTGGCTTTCAGCTATATGGATGATGTGGTATTGGGGGCCAAGTCTGTACAGCATCGTGA
AlTrpLeuSerAlaIleTrpMetMetTrpTyrTrpGlyProSerLeuTyrSerIleValS
GTCCCTTTATACCGCTGTTACCAATTTTCTTTTGTCTCTGGGTATACATTTAACGAATTC
ErProPheIleProLeuLeuProIlePhePheCysLeuTrpValTyrIleEnd
CAAGCTGAAACAATTCAAAGGTTTTCAAATCAATCAAGAACTTGTCTCTGTGGCTGATCC
AAACTACAAATTTATGCATTGTCTGCCAAGACATCAAGAAGAAGTTAGTGATGATGTCTT
TTATGGAGAGCATTCCATAGTCTTTGAAGAAGCAGAAAACAGATTATATGCAGCTATGTC
TGCCATTGATATCTTTGTTAATAATAAAGGTAATTTCAAGGACTTGAAATAATCCTTCTT
TCGTGTTCTTAATAACTAATATATAAATACAGATATAGATGCATGAATAATGATATACAT
TGATTATTTTGCAATGTCAATTAAAAAAAAAAAATGTTAGTAAAACTATGTTACATTCCA
AGCAAATAAAGCACTTGGTTAAACGAAATTAACGTTTTTAAGACAGCCAGACCGCGGTCT
AAAAATTTAAATATACACTGCCAACAAATTCCTTCGAGTTGTCCAATTTCACCACTTTTA
TATTTTCATCAACTTCAGCAGATTCAACCTTCTCACATAGAACATTGGAATAAACAGCCT
TAACACCACTTTCAAGTTTGCACAGCGTAATATGAGGAATTTTGTTTTGACAACACAACC
CTTTAATTTTCTCATTGTTTTCATCAATTATGCATCCATCTTTATCTTTAGACAGTTCCA
CTACAATAGCAATAGTTTTTTCATCCCAACATAGTTTTTCGAGCCTAAAATTCAGTTTGT
CGGTCGTTTTACCTGCGTATTTTGGTTATTACCAGAGCCTTGTGCATTTTCTATGCGGT
TGTTATTGTACTCCGTTATCTGGTCAGTGTATCTGTTACAATATGATTCCACAACTTTTT
TGCCTCTTTTTCACGGGACGACATGACATGACCTAATGTTATATGAAGTTCCTTCTGAAC
TTTTCCACTAGCTAGTAAATGCTTGAATTTCTCAGTCAGCTCTGCATCGCTAGCAATACA
CCTCTTGACCAATCAATAATTTCATCGTAGTTTTCTATTTAGCTGAGATATATGTAGGT
TTAATTAACTTAGCGTTTTTTGTTGATTATTGTTGCCTTTACCAACTATTTTTCTCACAG
TAGGTTTGTAATCTAAGCTCCTTCTGAACGCTGTCTCAATTTCATCATCTTTCGGGATCT
CTGGTACCAAAATTGGATAAGCTT

1. An immunogenic composition comprising:
a) an immunogenic particle RTS, S and/or
b) an immunogenic particle derived from the CS protein of one or more P. vivax strains and S antigen from Hepatitis B and optionally unfused S antigen, or
c) an immunogenic particle comprising RTS, CSV-S and optionally unfused S antigen, and
d) a stabilizing agent comprising a stabilizing agent with at least one thiol functional group, or mixtures thereof.

2. The immunogenic composition of claim 1 where in the stabilizing agent is N-acetyl cysteine, monothioglycerol, cysteine, reduced glutathione and sodium thioglycolate or mixtures thereof.

3. The immunogenic composition of claim 2, wherein the stabilizing agent is monothioglycerol, cysteine or a mixture thereof.

4. The immunogenic composition of claim 1, wherein the component is a liquid formulation.

5. The immunogenic composition of claim 4, wherein the pH of the liquid formulation is about 6.5 to 7.2.

6. The immunogenic composition of claim 1, wherein the formulation is lyophilized

7. The immunogenic composition of claim 1, wherein the stabilising agent is cysteine and is present in the range 0.1 and 1.0% w/w.

8. The immunogenic composition of claim 1, wherein the stabilising agent is monothioglycerol, which is present in the formulation in the range 0.01 to 1% w/w.

9. The immunogenic composition of claim 1, wherein the component is stored in a glass vial.

10. The immunogenic composition of claim 9, wherein the glass vial is amber.

11. The immunogenic composition of claim 9, wherein the glass vial is siliconised.

12. The immunogenic composition of claim 9, wherein the glass vial is un-siliconised.

13. The immunogenic composition of claim 1, wherein said component contains the elements for one dose for injection excluding adjuvant components.

14. The immunogenic composition of claim 13, wherein the one dose comprises 25 μg of RTS,S.

15. The immunogenic composition of claim 14, which further comprises 2.25 mg of sodium chloride.

16. The immunogenic composition of claim 14, which further comprises 125 μg of monothioglycerol.

17. The immunogenic composition of claim 1, which further comprises 250 μL of water for injection.

18. The immunogenic composition of claim 1, wherein said component contains the elements for 2 doses for injection excluding adjuvant components.

19. The immunogenic composition of claim 1, wherein the one dose comprises 50 μg of RTS, S.

20. The immunogenic composition of claim 19, which further comprises 4.5 mg of sodium chloride.

21. The immunogenic composition of claim 19, which further comprises 250 μg of monothioglycerol.

22. The immunogenic composition of claim 1, which further comprises 500 μL of water for injection.

23. The immunogenic composition of claim 1, which further comprises a further malaria antigen.

24. The immunogenic composition of claim 23, wherein the further malaria antigen is derived from P. falciparium and/or P. vivax wherein the antigen is selected from the group consisting of DBP, PvTRAP, PvMSP2, PvMSP4, PvMSP5, PvMSP6, PvMSP7, PvMSP8, PvMSP9, PvAMA, RBP or fragment thereof, PfEMP-I, Pfs 16 antigen, MSP-I, MSP-3, LSA-I, LSA-3, AMA-I and TRAP, EBA, GLURP, RAPI, RAP2, Sequestrin, PO32, STARP, SALSA, PfEXP1, Pfs25, Pfs28, PFS27/25, Pfs48/45, Pfs230 and their analogues in other Plasmodium spp.

25. The immunogenic composition of claim 1 further comprising an adjuvant selected from the group consisting of:
a. an oil in water formulation comprising QS21 and 3D-MPL, or
b. a liposomal formulation comprising QS21 and 3D-MPL.

26. A process for the preparation of the immunogenic composition of claim 1, comprising expressing a DNA sequence encoding the protein in a suitable host, recovering the product and mixing the recovered product with a stabilizing agent.

27. (canceled)

28. (canceled)

29. A method of treating or preventing Plasmodium infections in a subject comprising administering to a subject in need thereof an effective amount of the immunogenic composition of claim 1.


SEQUENCE LISTING (25 sequences)

SEQ ID NO: 1 - protein, 5 aa, Plasmodium vivax
KLKQP

SEQ ID NO: 2 - protein, 6 aa, Plasmodium vivax
CSVTCG

SEQ ID NO: 3 - protein, 9 aa, Plasmodium vivax
GDRAAGQPA

SEQ ID NO: 4 - protein, 9 aa, Plasmodium vivax
GDRADGQPA

SEQ ID NO: 5 - protein, 9 aa, Plasmodium vivax
GDRADGQAA

SEQ ID NO: 6 - protein, 9 aa, Plasmodium vivax
GNGAGGQPA

SEQ ID NO: 7 - protein, 9 aa, Plasmodium vivax
GDGAAGQPA

SEQ ID NO: 8 - protein, 9 aa, Plasmodium vivax
GDRAAGQAA

SEQ ID NO: 9 - protein, 9 aa, Plasmodium vivax
GNGAGGQAA

SEQ ID NO: 10 - protein, 9 aa, Plasmodium vivax
ANGAGNQPG

SEQ ID NO: 11 - protein, 12 aa, Plasmodium vivax
GGNAANKKAEDA

SEQ ID NO: 12 - DNA, 771 bp, Artificial Sequence
  Nucleotide sequence of the hybrid protein CSV of Plasmodium Vivax (optimised for expression in E. Coli)
acacattgcggacataatgtagatttatctaaagctataaatttaaatggtgtaaacttcaataacgtagacgctagttcactcggggctgcgcacgtaggtcagtctgctagcagggggcgcggtctcggggaaaacccagacgacgaagaaggtgatgctaaaaagaaaaaggacggtaaaaaagcggaaccaaaaaatccaagggaaaataaattaaaacagcccggggatcgcgcggatggtcaagcggcgggtaatggggcggggggtcaaccagcgggggatcgcgcggctggtcagccagcgggggatcgcgcggctggtcagccagcgggggatggtgcggctggccaaccagcgggggatcgcgcggatggtcagccagcgggggatcgcgcggatggtcaaccagccggtgatcgcgcggctggccaagcggccggtaatggggcggggggtcaagcggccgcgaacggagcggggaaccagccaggcggcggtaacgctgcgaataaaaaagcggaagatgcgggtggtaacgcgggcggtaatgcgggcggccaaggtcagaacaacgaaggggctaatgcaccaaacgaaaaatctgtcaaagaatatctcgataaagtccgcgctacagtagggacagaatggacgccatgctctgtaacatgtggtgtcggggtacgcgtgcgccgccgtgtcaatgcggctaacaaaaaaccagaagatctcacgttaaatgatctcgaaacggatgtctgcaca

SEQ ID NO: 13 - protein, 257 aa, Artificial Sequence
  Amino acid sequence of the hybrid protein CSV of Plasmodium Vivax
THCGHNVDLSKAINLNGVNFNNVDASSLGAAHVGQSASRGRGLGENPDDEEGDAKKKKDGKKAEPKNPRENKLKQPGDRADGQAAGNGAGGQPAGDRAAGQPAGDRAAGQPAGDGAAGQPAGDRADGQPAGDRADGQPAGDRAAGQAAGNGAGGQAAANGAGNQPGGGNAANKKAEDAGGNAGGNAGGQGQNNEGANAPNEKSVKEYLDKVRATVGTEWTPCSVTCGVGVRVRRRVNAANKKPEDLTLNDLETDVCT

SEQ ID NO: 14 - protein, 9 aa, Plasmodium vivax
ANGAGDQPG

SEQ ID NO: 15 - DNA, 771 bp, Artificial Sequence
  Nucleotide sequence for the hybrid protein CSV of Plasmodium vivax (optimised for expression in yeast)
acccattgtggtcacaatgtcgatttgtctaaggccattaacttgaacggtgttaatttcaacaacgtcgatgcttcttctttaggtgccgctcatgttggtcaatctgcttcaagaggtagaggtttaggtgaaaacccagacgacgaagaaggtgacgctaagaagaagaaggacggtaagaaggccgaaccaaagaacccaagagaaaacaagttgaaacaaccaggtgacagagccgacggacaagcagctggtaatggtgctggaggtcaaccagctggtgacagagctgccggtcagcctgctggtgatagagctgctggacaacctgctggagacggtgccgccggtcaacctgctggtgatagagcagacggacaaccagctggtgaccgtgctgacggacagccagccggcgatagggctgcaggtcaagccgctggtaacggtgccggtggtcaagctgctgctaacggtgctggtaaccaaccaggtggtggtaacgctgccaacaagaaagctgaagacgctggtggtaatgctggaggtaatgcaggtggtcagggtcaaaacaacgaaggtgctaacgctccaaacgaaaagtctgttaaggaatacttagataaggttagagctactgtcggtactgaatggactccatgttctgttacttgtggtgtcggtgttagagttagaagaagagttaacgccgctaacaagaagccagaagacttgactctaaacgacttggaaactgacgtttgtact

SEQ ID NO: 16 - DNA, 1485 bp, Artificial Sequence
  Nucleotide sequence for the hybrid fusion protein CSV-S
atgatggctcccgggacccattgtggtcacaatgtcgatttgtctaaggccattaacttgaacggtgttaatttcaacaacgtcgatgcttcttctttaggtgccgctcatgttggtcaatctgcttcaagaggtagaggtttaggtgaaaacccagacgacgaagaaggtgacgctaagaagaagaaggacggtaagaaggccgaaccaaagaacccaagagaaaacaagttgaaacaaccaggtgacagagccgacggacaagcagctggtaatggtgctggaggtcaaccagctggtgacagagctgccggtcagcctgctggtgatagagctgctggacaacctgctggagacggtgccgccggtcaacctgctggtgatagagcagacggacaaccagctggtgaccgtgctgacggacagccagccggcgatagggctgcaggtcaagccgctggtaacggtgccggtggtcaagctgctgctaacggtgctggtaaccaaccaggtggtggtaacgctgccaacaagaaagctgaagacgctggtggtaatgctggaggtaatgcaggtggtcagggtcaaaacaacgaaggtgctaacgctccaaacgaaaagtctgttaaggaatacttagataaggttagagctactgtcggtactgaatggactccatgttctgttacttgtggtgtcggtgttagagttagaagaagagttaacgccgctaacaagaagccagaagacttgactctaaacgacttggaaactgacgtttgtactcccgggcctgtgacgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaa

SEQ ID NO: 17 - protein, 494 aa, Artificial Sequence
  Amino acid sequence for the hybrid fusion protein CSV-S
MMAPGTHCGHNVDLSKAINLNGVNFNNVDASSLGAAHVGQSASRGRGLGENPDDEEGDAKKKKDGKKAEPKNPRENKLKQPGDRADGQAAGNGAGGQPAGDRAAGQPAGDRAAGQPAGDGAAGQPAGDRADGQPAGDRADGQPAGDRAAGQAAGNGAGGQAAANGAGNQPGGGNAANKKAEDAGGNAGGNAGGQGQNNEGANAPNEKSVKEYLDKVRATVGTEWTPCSVTCGVGVRVRRRVNAANKKPEDLTLNDLETDVCTPGPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 18 - DNA, 3509 bp, Artificial Sequence
  Nucelotide sequence for an RTS expression cassette
aagcttaccagttctcacacggaacaccactaatggacacaaattcgaaatactttgaccctattttcgaggaccttgtcaccttgagcccaagagagccaagatttaaattttcctatgacttgatgcaaattcccaaagctaataacatgcaagacacgtacggtcaagaagacatatttgacctcttaactggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaattagccttttaattctgctgtaacccgtacatgcccaaaatagggggcgggttacacagaatatataacatcgtaggtgtctgggtgaacagtttatccctggcatccactaaatataatggagctcgcttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctgtaaatctatttcttaaacttcttaaattctacttttatagttagtcttttttttagttttaaaacaccaagaacttagtttcgaataaacacacataaacaaacaaaatgatggctcccgatcctaatgcaaatccaaatgcaaacccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaataaaaaattctatttcaactgaatggtccccatgtagtgtaacttgtggaaatggtattcaagttagaataaagcctggctctgctaataaacctaaagacgaattagattatgaaaatgatattgaaaaaaaaatttgtaaaatggaaaagtgctcgagtgtgtttaatgtcgtaaatagtcgacctgtgacgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcgcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaacgaattccaagctgaaacaattcaaaggttttcaaatcaatcaagaacttgtctctgtggctgatccaaactacaaatttatgcattgtctgccaagacatcaagaagaagttagtgatgatgtcttttatggagagcattccatagtctttgaagaagcagaaaacagattatatgcagctatgtctgccattgatatctttgttaataataaaggtaatttcaaggacttgaaataatccttctttcgtgttcttaataactaatatataaatacagatatagatgcatgaataatgatatacattgattattttgcaatgtcaattaaaaaaaaaaaatgttagtaaaactatgttacattccaagcaaataaagcacttggttaaacgaaattaacgtttttaagacagccagaccgcggtctaaaaatttaaatatacactgccaacaaattccttcgagttgtccaatttcaccacttttatattttcatcaacttcagcagattcaaccttctcacatagaacattggaataaacagccttaacaccactttcaagtttgcacagcgtaatatgaggaattttgttttgacaacacaaccctttaattttctcattgttttcatcaattatgcatccatctttatctttagacagttccactacaatagcaatagttttttcatcccaacatagtttttcgagcctaaaattcagtttgtcggtcgttttacctgcgtattttggttattaccagagccttgtgcattttctatgcggttgttattgtactccgttatctggtcagtgtatctgttacaatatgattccacaacttttttgcctctttttcacgggacgacatgacatgacctaatgttatatgaagttccttctgaacttttccactagctagtaaatgcttgaatttctcagtcagctctgcatcgctagcaatacacctcttgaccaatcaataatttcatcgtagttttctatttagctgagatatatgtaggtttaattaacttagcgttttttgttgattattgttgcctttaccaactatttttctcacagtaggtttgtaatctaagctccttctgaacgctgtctcaatttcatcatctttcgggatctctggtaccaaaattggataagctt

SEQ ID NO: 19 - protein, 427 aa, Artificial Sequence
  Predicted translation product of the RTS-HBsAg hybrid protein
MMAPDPNANPNANPNANPNANPNANPNANPNANPNANPNANPNANPNANPNANPNANPNANPNANPNANPNANPNANPNANPNKNNQGNGQGHNMPNDPNDPNRNVDENANANNAVKNNNNEEPSDKHIEQYLKKIKNSISTEWSPCSVTCGNGIQVRIKPGSANKPKDELDYENDIEKKICKMEKCSSVFNVVNSRPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 20 - DNA, 20 bp, Artificial Sequence
  CpG oligonucleotide - CpG1826
tccatgacgttcctgacgtt

SEQ ID NO: 21 - DNA, 18 bp, Artificial Sequence
  CpG oligonucleotide - CpG 1758
tctcccagcgtgcgccat

SEQ ID NO: 22 - DNA, 30 bp, Artificial Sequence
  CpG oligonucleotide
accgatgacgtcgccggtgacggcaccacg

SEQ ID NO: 23 - DNA, 24 bp, Artificial Sequence
  CpG oligonucleotide - CpG 2006
tcgtcgttttgtcgttttgtcgtt

SEQ ID NO: 24 - DNA, 20 bp, Artificial Sequence
  CpG oligonucleotide - CpG 1668
tccatgacgttcctgatgct

SEQ ID NO: 25 - DNA, 22 bp, Artificial Sequence
  CpG oligonucleotide - CpG 5456
tcgacgttttcggcgcgcgccg